(19) United States
(12) Patent Application Publication
(10) Pub. No.: US 2026/0283800 A1
(43) Pub. Date: Sep. 24, 2026

(54) DEVICE FOR HEART REPAIR

(71) Applicant: CARDIOMECH AS, Oslo (NO)

(72) Inventor: Hans Emil HIORTH, Trondheim (NO)

(21) Appl. No.: 19/490,023

(22) PCT Filed: Jun. 6, 2024
(86) PCT No.: PCT/EP2024/065616

§ 371 Date: Dec. 4, 2025

(30) Foreign Application Priority Data
Jun. 8, 2023 (GB) 2308590.5

(51) Int. Cl. A61F 2/24; A61B 17/00; A61B 90/00

(52) U.S. Cl. CPC A61F 2/2466; A61B 17/00234; A61B 90/03; A61F 2/2457; A61F 2/246; A61F 2/2463; A61B 2017/00243; A61B 2017/00407; A61B 2090/034; A61F 2220/0008; A61F 2220/0075; A61F 2230/0004

(57) ABSTRACT

Disclosed herein are catheter devices for implanting an anchor system in soft body tissue and methods of using said catheter devices for repair of soft body tissue. The catheter device (2000) comprises: a gripper device (2006) for grasping soft body tissue (12), wherein the gripper device (2030 ) is configured to move between a closed configuration in which the soft body tissue (12) is grasped and an opened configuration in which the soft body tissue (12) is not grasped; a gripper control member (2040) configured to move the gripper device (2006) between the closed configuration and the opened configuration; and an anchor system deployment mechanism configured to deploy the anchor system (200) into the soft body tissue (12) when the soft body tissue (12) is grasped by the gripper device (2006). The gripper device (2006) comprises a gripper lock mechanism (2050) configured to releasably lock the gripper device (2006) in the closed configuration during deployment of the anchor system (200).

The present invention relates to a catheter device for implanting an anchor system in soft body tissue and a method of use of the catheter device for repair of soft body tissue, and in particular, but not exclusively, to catheter devices for repair of the heart by implanting such an anchor system in the heart valve leaflet to secure an artificial chordae line.

The chordae tendineae are cord-like tendons that connect the papillary muscles to the tricuspid valve and the mitral valve in the heart. The valves consist of leaflets that open and close with the beating of the heart in order to control blood flow and blood pressure within the heart.

Mitral valve disease presents an important challenge to cardiac surgeons and cardiologists. Mitral regurgitation has become the leading pathophysiological condition of the mitral valve in the developed world. One of the most important causes of regurgitation is prolapse of one of the mitral leaflets. The pathological abnormality that requires repair is rupture or other degenerative changes of the chords, leaflet or other related structures. When the chord(s) remain intact, the mitral leaflets open and close synchronously and in a fashion that prevents leakage of the valve. The normal chords can rupture acutely, causing acute decompensation in the form of heart failure. This usually results in an emergency condition requiring rapid intervention. Damage to the chord(s) can also occur more slowly including rupturing or elongation due to degenerative processes, causing the mitral valve to develop leaks or regurgitation.

Surgical repair of the mitral valve has become relatively standardized, using resection of the prolapsed leaflet and/or implantation of new, artificial chordae lines to control leaflet motion. In addition, a mitral ring is frequently placed to shrink the size of the mitral valve annulus. Surgical replacement of ruptured or elongated chords is highly effective in eliminating or minimizing mitral valve regurgitation. The procedure is presently performed with open heart surgery techniques. This requires use of cardiopulmonary bypass and arresting of the heart. This surgical approach, although working well, is a highly invasive procedure which can cause serious complications, long hospital stays and substantial expense. Consequently, a less invasive approach would be preferable. Similarly, a less invasive approach would also be preferable for treatment of the tricuspid valve which, analogously to the mitral valve, may suffer tricuspid valve disease.

Insertion of mitral leaflet chords has been done using a minimally invasive surgical approach entering the heart through its apex. The technique, was developed by the company Neochord Inc. and is described, for example, in WO2012/167120, but still requires a surgical incision and the chords do not get inserted in the papillary muscles where they normally should be fixed.

WO2008/101113 describes another example of a system for repair of the heart, including implantation of artificial chordae lines. In the described method an anchor can be attached to the papillary muscle and is coupled to the leaflet of the mitral valve by an artificial chordae line, a suture and a clip. The clip allows for adjustment of the length of the artificial chordae line. A complex multi-stage process is required to implant the papillary anchor and the suture and join them together. The papillary anchor is formed of a memory metal such as nitinol and has a ‘flowered’ shape with sharp ‘petals’ for hooking the anchor to body tissue. The flowered shape is flattened into a tube shape and held in a tube that is passed into the heart. The tube and anchor are then pressed against the papillary muscle and the anchor is pushed out of the tube so that the petals pierce the muscle and fold outward through the muscle to provide a secure coupling of the anchor to the muscle tissue. In a subsequent surgical procedure, an artificial chordae line may be attached to the anchor. Then in a further step, the suture is attached to the leaflet and this suture is joined to the chord by the clip. The suture is attached to the leaflet by locating a vacuum port near to the leaflet and pulling it into the vacuum port where it can be pierced.

It will be appreciated that this technique, whilst avoiding open heart surgery, still requires a sequence of relatively complex steps. The number of steps required increases the risk. Furthermore, the complexity of the device means that parts implanted within the body are at risk of coming loose and injuring the patient by embolization. In particular, the clip could come loose from the anchors. It is also thought that the use of a suture with an additional clip, as proposed, may not effectively repair the heart valve since it will not closely simulate a natural chord.

In an earlier patent application, WO2016/042022, the present applicant disclosed a catheter device for implanting an artificial chordae line to repair a heart valve. The catheter device of WO2016/042022 includes a mechanical gripper device for grasping the leaflet of the heart valve, with a leaflet anchor housed in the gripper. The leaflet anchor can be formed from a flexible material, such as nitinol, with a grapple hook shape in an unfolded configuration, and being able to deform elastically into the folded configuration, for example when constrained within a leaflet anchor channel in the gripper device. The hooks are straightened out when the leaflet anchor is in the folded configuration. When the leaflet is grasped by the gripper device then the leaflet anchor can be pushed out of the gripper to drive the hooks though the leaflet whilst they return elastically to the unfolded configuration, thereby securing the leaflet anchor in the leaflet.

The device described in WO2016/042022 also uses a papillary anchor with a broadly similar arrangement of foldable hooks. The papillary anchor is held within a tube of the catheter device in a folded configuration and can be pushed out of the tube with the hooks being driven into the heart wall whilst they return elastically to the unfolded configuration, thereby securing the papillary anchor to the muscle. The papillary anchor includes a locking ring acting as a locking mechanism for clamping an artificial chordae line when no force is applied. The locking ring maybe elastically deformed to release the line from the locking mechanism for adjustment of the length of the chordae line.

In another earlier patent application, WO2020/109596, the present applicant disclosed further refinements to the catheter device disclosed in WO2016/042022, and new developments related thereto. One area of refinement focussed on the functionality of the mechanical gripper device.

Whilst the devices of WO2016/042022 and WO2020/109596 provided a significant advance in this field, it has been found that further refinement of the design may be advantageous. The present disclosure relates to new features building on the design of the device disclosed in WO2016/042022 and WO2020/109588 in various respects.

It is an objective of the present invention to provide an improved catheter device for implanting an anchor system in soft body tissue, and more preferably in heart tissue.

Viewed from a first aspect of the present invention, there is provided a catheter device for implanting an anchor system in soft body tissue. The catheter device comprises: a gripper device for grasping soft body tissue, wherein the gripper device is configured to move between a closed configuration in which the soft body tissue is grasped and an opened configuration in which the soft body tissue is not grasped; a gripper control member configured to move the gripper device between the closed configuration and the opened configuration; and an anchor system deployment mechanism configured to deploy the anchor system into the soft body tissue when grasped by the gripper device; wherein the gripper device comprises a gripper lock mechanism configured to releasably lock the gripper device in the closed configuration during deployment of the anchor system.

During implantation of the anchor system in the soft body tissue, the deployment force used to deploy the anchor system can cause the gripper device to inadvertently open. If the gripper device inadvertently opens, it may malfunction. For example, this may result in the anchor system being incorrectly placed in the soft body tissue after insertion, or unnecessary trauma being experienced by the soft body tissue during implantation. To prevent the gripper device from inadvertently opening, greater force can be applied via the gripper control member to keep the gripper device closed. However, this can increase trauma to the soft body tissue as it is grasped, and can also increase a risk of malfunction of the delivery device during operation of the gripper device.

The present invention therefore provides a catheter device comprising a gripper lock mechanism that is configured to releasably lock the gripper device in the closed position during deployment of the anchor system. By using a gripper lock mechanism to keep the gripper device closed during deployment, rather than the gripper control member itself or directly, a lower force may be required to be applied to the gripper device to keep the gripper arm closed. This is because the gripper lock mechanism may provide a constant amount of force (i.e. a locking force) resisting opening of the gripper device, compared to the gripper control member that opens and closes the gripper device. In other words, the gripper lock mechanism can increase the load required to open the gripper device when otherwise closed, thereby reducing the force required to be provided by the gripper control member to resist the gripper device inadvertently opening during deployment of the anchor system. This may improve the stability of the soft body tissue when grasped by the gripper device during deployment of the anchor system.

The gripper device is preferably a mechanical gripper device.

The catheter device may comprise a housing section, wherein the housing section extends from a proximal end to a distal end.

The gripper device may comprise a gripper arm coupled to the housing section.

The gripper arm may be configured to move between a closed configuration in which the soft body tissue is grasped and an opening configuration in which the soft body tissue is not grasped. The gripper control member may be configured to move the gripper arm between the closed configuration and the opened configuration. The gripper lock mechanism may be configured to releasably lock the gripper arm in the closed configuration during deployment of the anchor system.

Preferably, the gripper arm is rotatably coupled to the housing section, and the gripper arm is configured to rotate to move between the closed configuration and the opened configuration. In other arrangements, however, the gripper arm could be slidably coupled to the housing section, and the gripper arm could be configured to translate or slide to move between the closed configuration and the opened configuration.

The gripper control member may be a gripper control wire, a gripper control rod or a gripper control piston. The gripper control member may be any suitable member for moving the gripper arm between the opened and closed configurations.

The gripper device, or particularly the gripper arm of said device, may generally comprise a gripper surface facing the housing section, wherein the soft body tissue is grasped between this gripper surface and the housing section. The gripper arm may comprise the gripper surface, which is configured to be moved away from the housing section and a lever portion. The lever portion may be connected to the gripper control member, for example directly or indirectly.

The gripper device, and particularly the gripper arm of said device, may comprise a hinge. The gripper device may be movable about the hinge. For example, the gripper arm may be rotatably coupled to the housing section about the hinge. The hinge may be located between the gripper surface and the lever portion. The gripper arm may be hinged to the housing section such that actuation of the lever portion rotates the gripper arm about the hinge.

The gripper device, and particularly the gripper arm of said device, may be joined to the housing section using a pin joint, the pin forming an axis of rotation of the gripper arm. The pin joint may be a revolute joint or a hinge joint, i.e. comprising intermeshing features with a pin or cylindrical member joining said members, the pin forming the axis of rotation for the joint.

The gripper control member may be configured to operate the gripper lock mechanism. That is, the gripper control member can be configured to operate the gripper device (e.g. the gripper arm) and the gripper lock mechanism.

By providing a gripper control member that can be used to both open and close the gripper device and to engage the gripper lock mechanism, the design of the catheter device may be made simpler and more compact. For example, operating the gripper device using just a single control input may prevent malfunctions associated with multiple control inputs occurring, such as a twisting or tangling of control wires. The catheter device may be made more compact or simple because only a single gripper control wire lumen may be required to provide complete operation of the gripper device.

The catheter device may comprise a single gripper control member to operate the gripper device and the gripper lock mechanism. The catheter device may comprise a single lumen housing the gripper control member.

The gripper lock mechanism may be configured to releasably lock the gripper device in one of a plurality of positions when in the closed configuration. Each position is of a different, or may define a varying, distance or separation for the gripper device to accommodate the soft body tissue, for example between the gripper arm and the housing section.

By providing a gripper lock mechanism that enables the gripper device to be locked at a plurality of positions when in the closed configuration, soft body tissue of varying size and thickness can be stably grasped by the gripper device.

The gripper lock mechanism may be configured to lock the gripper arm at or towards an end of the lever portion distal to a centre of rotation of the gripper arm. Locking the gripper arm at a distance away from the centre of rotation of the gripper arm may provide a greater moment of locking force opposing the deployment force applied to the gripper arm.

The gripper lock mechanism may comprise a link member connecting the gripper control member to the gripper arm, and a lock member fixedly attached to the housing section; wherein the link member is configured to engage the lock member to releasably lock the gripper arm.

The link member may connect to the gripper arm at a location distal to the centre of rotation of the gripper arm, e.g. at or towards an end of the lever portion.

The link member may comprise a first locking portion, and the lock member may comprise a second locking portion. The first locking portion and the second locking portion are complementary, or are configured to mate, such that the link member may engage the lock member.

The link member is used to communicate control inputs from the gripper control member to the gripper arm and to enable releasable locking of the gripper arm when in the closed position. The link member may be located within a groove or a channel. The link member may be constrained by the groove or channel such that its motion is within a desired plane of motion.

The lock member is used to selectively prevent motion of the link member. The lock member is fixed to the housing section such that, when the link member engages the lock member, the link member and the gripper arm cannot move relative to the housing section. That is, the gripper arm remains locked in place.

The lock member may comprise a large rack and a small rack. The large rack may be fixed to the housing section. The small rack may be fixed to the large rack. The small rack may define or comprise the second locking portion.

Alternatively, the lock member may comprise a single structure both fixed to the housing section and defining or comprising the second locking portion.

The lock member may define an opening configured to facilitate a coupling of a proximal part of the housing section to a distal part of the housing section. The opening may facilitate a hinged coupling.

The lock member may comprise a ratchet gear rack; and the link member may comprise a protrusion configured to engage with the ratchet gear rack. That is, the gripper lock mechanism may be a ratchet gear rack mechanism.

The ratchet gear rack generally comprises a plurality of toothed members or protrusions. The ratchet gear rack extends along a surface of the lock member.

The protrusion extends from the link member, and may be a toothed member. The protrusion is complementary to the ratchet gear rack. The link member may function as a pawl. That is, the protrusion may be configured to backstop or prevent motion of the link member when the link member engages the ratchet gear rack.

The ratchet gear rack may be configured to prevent motion of the link member moving the gripper arm from the closed configuration to the opened configuration, or alternatively between the closed configuration and the opened configuration altogether.

The link member may be configured to engage the lock member at a plurality of positions. Each position may control the separation of the gripper arm from the housing section when in the closed configuration. For example, each step or toothed member in the ratchet gear rack may define a position or separation of the gripper arm relative to the housing section. Accordingly, the manner in which, or the position at which, the link member engages the lock member may control the separation of the gripper arm relative to the housing section.

The link member may comprise a plurality of protrusions.

By providing a plurality of protrusions, the link member may be able to more readily engage the lock member. Accordingly, providing a plurality of protrusions may improve the resolution of the control of the gripper lock mechanism, since there exists a plurality of stable positions for the link member to engage the lock member.

A plurality of the protrusions of link member may be configured to simultaneously engage the lock member. By providing a plurality of protrusions configured to simultaneously engage the lock member, the link member may be configured to engage the lock member with multiple protrusions. This may increase the locking force of the gripper lock mechanism.

In some arrangements, the link member may comprise a sprag, or a plurality of sprags. The lock member may define a race. The gripper lock mechanism may accordingly comprise a sprag clutch mechanism.

The one or more sprags may be configured to engage the race. Owing to the shape of each of the one or more sprags, when the one or more sprags engage the race the link member may be prevented from moving the gripper arm from the closed configuration to the opened configuration. The sprags may experience a backstopping or be backstopped by the race. The link member is preferably fixed relative to the gripper control member in this arrangement, i.e. such that it does not move relative to the gripper control member. The link member may be integrally formed with the gripper lock member, in this configuration.

The position at which the one or more sprags engages the race may control a position or separation from the housing section at which the gripper arm is locked when in the closed configuration.

The material of the one or more sprags and the race may be selected so as to obtain a desired locking force. For example, the one or more sprags and the race may be made of steel. Alternatively, the race may be lined with a higher-friction material, such as a polymer, a rubber, or a rubber composite. The sprags may also be made of a higher-friction material, such as a polymer, a rubber, or a rubber composite. The one or more sprags and/or the race may be made of steel and may be coated in a layer of the one or more aforementioned higher-friction materials.

The link member may comprise a first end coupled to the gripper arm and a second end coupled to the gripper control member. The link member may be a separate component to the gripper control member, or the link member may be integrally formed with, i.e. as part of, the gripper control member.

The first end of the link member is generally movably coupled to the gripper arm, and is preferably rotatably coupled to the gripper arm. The gripper arm may comprise a pin or a protrusion, and the first end of the link member may comprise an opening. The opening may be configured to receive the pin.

The gripper control member may comprise a joint member. The joint member may be located at a distal end of the gripper control member. The link member may be coupled to the gripper control wire via the joint member. The joint member may be an integral part of the gripper control member, or may be a separate part fixed or attached to the gripper control member.

The first end of the link member may be rotatably coupled to the gripper arm, and the second end of the link member may be rotatably coupled to the gripper control member.

The joint member may comprise an opening and the second end may comprise an opening. The respective openings may be coupled via a pin extending therethrough.

Alternatively, the second end of the link member may be rotatably coupled to the joint member via a hinge joint. The second end of the link member may define a socket or cup configured to receive a cylinder member of the joint member. The cylinder member may be configured to engage the link member via a snap-fit engagement, or any other suitable mechanism that facilitates a rotational coupling.

The gripper lock mechanism may comprise an abutment portion. The abutment portion is configured to prevent the first end of the link member crossing an axis extending between the second end of the link member and the centre of rotation of the gripper arm when the gripper arm is moved between the closed configuration and the opened configuration.

The abutment portion may define a maximum separation, i.e. a most open position, of the gripper arm from the housing section. That is, the abutment portion may limit the range of motion of the gripper arm.

By providing an abutment portion, the gripper arm may be prevented from overextending. An overextension of the gripper arm could otherwise cause the gripper arm to lock out in the opened configuration. Accordingly, the provision of an abutment portion may facilitate a smoother motion of the gripper arm between the closed configuration and the opened configuration.

The abutment portion may be a protrusion extending from the first end of the link member, wherein the protrusion is configured to abut a shoulder region of the gripper arm when the gripper arm is in the opened configuration.

The abutment portion moves with the link member, since it is formed on, part of, or attached to, the link member. The shoulder region is proximate to the first end of the link member, and is preferably formed on a surface of the lever portion of the gripper arm. The abutment portion is configured to engage the shoulder region when the gripper arm is at a most open position in the opened configuration.

The abutment portion may be a post located in the housing section, wherein the post is configured to abut the link member when the gripper arm is in the opened configuration.

The abutment portion is fixed relative to the link member. The post may extend between opposite surfaces within the housing section, or may be a protrusion formed or fixed on a respective surface of the housing section. The link member is configured to engage the post when the gripper arm is at a most open position in the opened configuration.

In other arrangements, the abutment portion may be a protrusion extending from the second end of the link member. The abutment portion may be configured to abut the joint member, or a mating surface, of the gripper control member when the gripper arm is in the opened configuration.

In other arrangements, the link member may be integrally formed with the gripper control member. The link member may be rotatably relative to the gripper control member via a compliant joint or spring mechanism.

The link member may be biased to deform in a direction toward the gripper arm, i.e. such that the link member deforms away from an axis extending between the second end of the link member and the centre of rotation of the gripper arm when the gripper arm is moved between the closed configuration and the opened configuration. This may prevent overextension of the link member.

Alternatively, the first end of the link member may be rotatably coupled to the gripper arm, but instead the second end of the link member may be fixed to the gripper control member.

The link member is fixed to the gripper control member such that it does not move relative to the gripper control member. The link member may be integrally formed with the gripper control member. Alternatively, the link member may be fixedly coupled to the gripper control member, for example by gluing or welding. The link member may comprise a socket or recess configured to receive the wire guide member.

The first end of the link member may comprise an opening rotatably coupled to a pin of the gripper arm, wherein there is a clearance between the pin and the opening.

The opening is wider than the pin, such that there is a clear gap or clearance between the pin and the opening. Accordingly, the link member is configured to transfer a control input from the gripper control member to the gripper arm with a degree of play or backlash.

By providing an opening with clearance between itself and the pin, smoother control of the link member may be achieved when engaging the lock member.

The pin may comprise a cap or fixing portion such that the link member cannot disengage the gripper arm.

In some arrangements, the lock member may comprise a first rack portion and a second rack portion. The second rack portion may comprise or define the second locking portion, and the first rack portion may be fixed to the housing section. The second rack portion may be connected to the first rack portion by a compliant mechanism. The compliant mechanism may resiliently bias the second rack portion in the proximal direction, or such that it readily engages with the link member.

The lock member may be configured to deform when a force is applied by the gripper control member. The link member may be able to slide along, or move relative to, the lock member when the lock member is deformed. Such a mechanism may provide smoother control of the link member when engaging the gripper control mechanism.

In some arrangements, the link member may comprise a spring member extending from the link member, wherein the first locking portion is located at an end of the spring member.

A first end of the spring member may be fixed to the link member. The first locking portion may be located on, or at, the second end of the spring member, wherein the second end of the spring member is opposite to the first end.

Providing the first locking portion on the end of a spring member may introduce a degree of compliance that enables the first locking portion to more readily engage with the second locking portion.

The second end of the spring member may be movably coupled to the link member. The second end of the spring member may be coupled to the link member by a hinge joint, for example. Movably coupling the second end of the spring member to the link member may provide additional support to the first locking portion located at the end of the spring member, whilst also providing compliance to the first locking portion.

The anchor system deployment mechanism may be configured to hold and/or guide the anchor system during deployment into the soft body tissue. The anchor deployment mechanism may comprise an anchor deployment tube that holds and guides part(s) of the anchor system.

The anchor system deployment mechanism may be configured to deploy the anchor system out of the anchor system deployment mechanism in a direction extending from the proximal end of the catheter device toward the distal end of the catheter device.

The catheter device may comprise a linear-shaped rod for deployment of the anchor system from the anchor system deployment mechanism.

The catheter device may comprise the anchor system, wherein the anchor system is located within the anchor system deployment mechanism.

The anchor system deployment mechanism may define an anchor system tube. The anchor system may be arranged to be deployed from the anchor system tube.

An opening of the anchor system deployment mechanism may comprise an opening, wherein the anchor system is arranged to be deployed by pushing it out of an opening at the end of the anchor system tube. The opening is arranged to be in contact with the soft body tissue during deployment.

The anchor system is preferably a soft tissue anchor system, i.e. an anchor system for implantation in soft body tissue.

The soft tissue anchor system may comprise a U-shaped fabric body comprising a base portion and at least two arm portions extending from the base portion, wherein each arm portion is configured to collapse in folds towards the base portion such that, in use, the soft body tissue is sandwiched between the base portion and each of the arm portions.

By providing a fabric body comprising two or more arm portions extending from the same base, the anchor system may be able to contact a greater surface area of body tissue when implanted, and hence may have improved stability and/or improved tissue ingrowth upon implantation. Further, by providing the collapsible arm portion(s) attached via a base portion, the base portion itself may further be able to provide a greater lateral surface by which to connect the anchor system and the leaflet upon implantation. A U-shaped anchor system, i.e. with at least two arm portions, may provide enhanced performance compared to the single arm portion.

Accordingly, a soft tissue anchor system as described above may have an improved securement strength compared to known anchor systems comprising a single arm portion and/or fixing member and/or using different design features.

Further, the use of a fabric body compared to a rigid body such as a metal body may reduce damage to the body tissue caused by the anchor during and/or after implantation. The fabric may better complement a surface of the body tissue that it contacts, thus spreading force exerted on the body tissue over a greater area and reducing trauma experienced by the body tissue at the site of implantation.

Collapsing of the arm portions in folds towards the base portion may be considered as a concertina motion. Accordingly, the arm portions may also be configured to concertina towards the base portion such that, in use, the body tissue is sandwiched between the base portion and the arm portions.

The fabric body, being formed of a fabric material, may be naturally disposed to fold and/or concertina because of the conformable nature of fabric. However, in some arrangements, the arm portions may comprise fold lines, narrowed sections, and/or weakened portions configured to aid in folding and/or concertinaing the arm portions.

In one arrangement, each arm portions may be configured to collapse in folds towards the base portion by action of a tension line threaded through the arm portions and the base portion. Where two arm portions are used then each can have its own tension line. Thus, the tension line may be advantageously configured to collapse the arm portion in folds towards the base portion when a tensile force is applied to the tension line. It will be appreciated that each tension line could act as implantable chords (i.e. the tension line may be the artificial line).

The tension line is preferably fixed to the arm portion at an end of the arm portion distal to the base portion. Thus, when a tensile force is applied to the tension line, the entirety of the arm portion may be configured to collapse in folds towards the base portion.

The location of the threading of the tensile line may facilitate the collapse of the arm portion in folds. For example, as the tension line is pulled, the tension line may draw holes in the arm portion through which the tension line is threaded together. Accordingly, the arm portion may be biased to fold in relation to each location the tension line is threaded through the arm portion.

Preferably, the anchor system is configured so that the tensile force is applied to a portion of the tension line(s) threaded through the base portion. This arrangement may facilitate the entire collapse of the arm portions.

The line may be threaded no more than three or four times through the arm portion and the base portion. This arrangement may facilitate the holes in the fabric body through which the line is threaded being further spaced apart, and/or a shorter fabric body being employed. Relative to the size of the anchor, each of these arrangements may provide wider folding portions of the fabric body that provide improved stability for the anchor system upon implantation. Using a shorter fabric body may also reduce the size of the overall soft tissue anchor system, thus improving its packaging within a delivery device/system for implantation.

The soft tissue anchor system may comprise the artificial line. That is, the fabric body may be in combination with the artificial line.

The artificial line may be fixedly joined to the line such that the relative positions of the line and the artificial line does not change.

The artificial line may be slidably joined to the portion of the tension line threaded through the base portion. The artificial line may be arranged to apply the tensile force to the tension line.

Alternatively, the artificial line may be slidably joined to the tension line by any suitable knot, for example a bridle knot. Alternatively, the artificial line may be slidably joined to the tension line by an intermediate member, such as an eyelet connected to the artificial line through which the tension line passes.

The artificial line may be joined to the fabric body via the base portion, using any suitable fastening arrangement.

The tension line and/or the artificial line may be formed of a suture material.

Each arm portion comprises an end cap fixed at an end of each arm portion distal to the base portion, and each end cap comprises an opening configured to engage a wire guide member for implanting the U-shaped fabric body in the soft body tissue.

By providing end caps comprising openings configured to engage a wire guide member for implanting the fabric body, the arm portions may be manipulated such that they can be implanted through the body tissue. For example, the wire guide members can push the arm portions in a direction of their engagement such that the arm portions pass through the body tissue.

Further, by using wire guide members which guide the arm portions through the body tissue, rather than a needle from which the arm portions are deployed, a hole in the body tissue through which each arm portion is passed need not be as large as a hole required by a needle or other conduit containing the arm portions to pass through. That is, the size of the openings is constrained by no more than the geometry of the fabric body. In comparison, the size of openings which are required for implantation via a needle will always be constrained by the size of the needle which is always larger than the member it is to deploy. This may reduce trauma at the site of implantation.

The catheter device may comprise the wire guide member for deploying the soft tissue anchor system.

The anchor system may comprise a tension line (e.g. as described above). The tension line may be fixed to the end cap and extends from a central portion thereof such that, in use, the end cap is configured to extend in a plane parallel to a surface of the body tissue when implanted in the soft body tissue and under tension of the line when it is passing through the tissue in a direction away from the surface thereof.

Each end cap may comprise a pointed tip. The pointed tip may be configured to pierce the body tissue and, for example, the pointed tip may pierce the body tissue when a motive force is applied to the end cap by the wire guide member.

The tip of the end cap may be located at and end of the end cap distal to the arm portion (i.e. at an end of the end cap distal to the opening configured to engage the wire guide member).

Each end cap may comprise an outer tubular member and an inner tubular member, wherein the inner tubular member is configured to be received by the outer tubular member. That is, the inner tubular member may be nested within, and/or concentric with the outer tubular member. The inner tubular member may define the opening configured to engage the wire guide member.

The outer tubular member and the inner tubular member may be configured to sandwich, crimp and/or clamp the distal end of a respective arm portion therebetween, thereby fixing the end cap to the arm portion. Adhesives may be additionally or alternatively employed, to fix the end cap to the arm portion.

The inner tubular member may comprise a flared inlet defining the opening configured to engage the wire guide member. The flared inlet will be understood to be a portion of the inner tubular member defining the opening, wherein a circumferential extent of the inner tubular member is increased relative to the rest of the inner tubular member.

The flared inlet may be configured to mate with a corresponding portion of the wire guide member. That is, the flared inlet may be complementary to a shape of the shoulder region, or bulge portion, of the piercing wire guide member. The angled face of the flared inlet may be complementary to the shoulder region, or bulge portion, of the wire guide member. The flared inlet may improve contact between the wire guide member and the end cap during implantation of the fabric body.

The outer tubular member may define the pointed tip. The inner tubular member may comprise a blind bore which receives the wire guide member.

The tension line may be fixed to the end caps. Each end cap may be configured to receive the tension line between the inner tubular member and the outer tubular member. The tension line may be fixed to each end cap by crimping, swaging, clamping, gluing and/or sandwiching the tension line between the inner tubular member and the outer tubular member. The tension line could also be fixed by stitching it or tying it to the tubular member.

The outer tubular member may comprise an opening formed in a side wall, the opening configured to receive the tension line. The opening may be formed in a central region of the end cap, and more preferably the opening may be formed towards the tip of the end cap.

Each end cap may be configured to extend collinearly with a respective arm portion during implantation and/or retrieval of the U-shaped fabric body. Each end cap may be configured to extend parallel to a plane of each fold of the respective arm portion when a tensile force is applied to the tension line.

As the arm portion may be fixed at an end of the end caps proximal to the arm portions, and as the tension line may be fixed at a central region and/or towards a tip of the end cap distal to the arm portion, tensile forces applied due to the arm portions and/or the tension lines may cause a torque to act on the end caps.

Accordingly, when the wire guide member engages the end cap, the end cap will straighten and extend collinearly with the arm portions. This may facilitate implantation of the arm portions of the fabric body in the body tissue. When the tension line experiences a tensile force and thus collapses the arm portions in folds, the tension line may turn the end caps to lie parallel with the folds of the arm portion, and hence parallel with a surface of the body tissue in which the arm portions fold against. This may facilitate securement of the arm portions to the body tissue, and may also minimise a profile of the end caps protruding from the body tissue during implantation. For example, the end cap may turn perpendicular to the tension line in a “T” configuration, with the tension line normal to the surface of the body tissue and the length of end cap parallel to the surface of the body tissue, preventing movement of the end cap through the body tissue.

The wire guide member may be the aforementioned linear-shaped rod.

Alternatively, the soft tissue anchor system may comprise an anchor having a number of hooks for engagement with the soft body tissue and having a folded position and an unfolded position, wherein the anchor is made of an elastic material such that it can be elastically deformed into the folded position by application of a constraining force, and will return to the unfolded position when no constraining force is applied.

The anchor having hooked formations may be a leaflet anchor as described in WO2016/042022 or WO2020/109596.

The gripper device may comprise a plurality of serrations for grasping the soft body tissue. The gripper arm itself may comprise a or the plurality of serrations.

The serrations may be located on a first portion of the gripper arm configured to face an opening of the anchor system deployment mechanism, and on a second portion of the gripper arm configured to face the housing section.

The serrations may increase an area of contact between the soft body tissue and the gripper device. Thus, by providing serrations surrounding the gripper device in this manner, the gripper device may provide an improved grasping contact between itself and the soft body tissue. For example, the serrations may effectively circumferentially surround the face of the gripper arm configured to contact the soft body tissue. Accordingly, the soft body tissue may be better supported by the gripper device when grasped.

The housing may also comprise a plurality of serrations, formed on a surface configured to face the gripper arm. Providing serrations on both gripper arm and the housing may further improve the force with which the gripper arm grasps the leaflet.

The serrations are preferably dulled so as to minimise trauma experienced by the soft body tissue when grasped. The serrations may be unevenly and/or evenly distributed undulations in the surface of the gripper arm and/or the housing of the catheter device. The serrations increase a surface area thereof compared to e.g. a flat/planar surface.

The gripper device may comprise an internal space configured to receive the anchor system during deployment of the anchor system. The internal space may be formed in the gripper arm.

The internal space may be configured to receive arm portion(s) and/or end cap(s) and the wire guide member(s) deployed from the anchor system deployment mechanism. The internal space may be open to a face of the gripper arm configured to contact the soft body tissue, and bounded by side walls of the gripper arm.

The internal space may facilitate a complete extension of an anchor deployment member, such as the linear rod or the wire guide member(s) such that the arm portion(s) and/or end cap(s) are completely passed through the leaflet, or hooked formations such that they are completely passed through the leaflet, and thus reliably implanted in the soft body tissue.

The internal space may comprise a first internal space configured to receive a first wire guide member or a first hooked formation and a second internal space configured to receive a second wire guide member or a second hooked formation. The openings of the first and second internal spaces may be separated by a gripping portion configured to contact the leaflet. Providing a gripping portion may increase the surface area of the soft body tissue in contact with the gripper arm, and thus the stability of the soft body tissue during implantation of multiple arm portions and/or end caps or hooked formations into the soft body tissue.

The anchor system deployment mechanism, or another part of a catheter device that holds the anchor deployment mechanism, may be configured to remove the wire guide member(s) and/or tension the tension line in order to place the arm portion(s) and/or end cap(s) into their final position, e.g. with the arm portion(s) folded and/or the end cap(s) turned to sit along the surface of the body tissue.

The anchor system may be held within a curtain or sheath in the catheter device, for example in the anchor system deployment mechanism. The sheath may be configured to deflect, or crumple, during deployment to aid deployment of the anchor system, particularly deployment of the soft tissue anchor system having a fabric body, from the catheter device. The sheath may reduce friction between the anchor system and the catheter device during deployment of the anchor system from the catheter device.

The sheath may be a thin tubular sheath.

Where the anchor system comprises a fabric body, the sheath may be used to hold the fabric body. Where the anchor system comprises tubular cap members/end caps, said members/caps may be held outside or within the sheath.

The sheath may be housed within the anchor system deployment mechanism.

The sheath may be attached or secured to the anchor system deployment mechanism, such that it does not release from the catheter device during deployment of the anchor system from the catheter device.

The anchor deployment mechanism may comprise an anchor deployment tube that holds and guides part(s) of the anchor system, such as the arm portion(s) and/or end cap(s), and wire guide member(s), or hooked formations.

In the case where multiple arm portions are used, such as with a U-shaped fabric body and two arm portions, or hooked formations are used, then the anchor deployment tube may comprises a pair of tubes, one for each arm portion and its respective wire guide member or each hooked formation, with a join between the tubes allowing for the connective bridge of fabric from the base portion between the arm portions to span between the tubes, or for a base of the anchor having hooked formations to span between the tubes. For example, there may be slots along the tubes for the connective bridge or base to slide along.

The sheath may be held within the anchor deployment tube, and may extend to the tubes housing the end caps/hooked formations.

The anchor system may be a leaflet anchor system, wherein the soft body tissue is a heart valve leaflet, and wherein the catheter device is for implanting the leaflet anchor system in the heart valve leaflet to secure an artificial chordae line.

The anchor system deployment mechanism may be arranged to implant the leaflet anchor system in the heart valve leaflet by piercing the leaflet from an atrial side of the leaflet. The leaflet may be a mitral valve leaflet or a tricuspid valve leaflet.

The previous catheter devices of WO2016/042022 and WO2020/109596 generally contemplated the implantation of the leaflet anchor system in the leaflet of the heart by piercing the leaflet from a ventricular side of the leaflet, rather than an atrial side of the leaflet. However, the present application recognises that a catheter device which implants a leaflet anchor system from an atrial side of the leaflet provides a number of advantages which may not be present when a leaflet anchor system is implanted from a ventricular side of the leaflet of the heart.

When implanted from a ventricular side of the leaflet, the leaflet anchor system may need to be located towards an edge of the leaflet in order to provide adequate support to the flailing leaflet. In contrast, when implanted from an atrial side of the leaflet, the leaflet anchor system may provide adequate support to the edge of the leaflet when the leaflet anchor system is implanted towards the edge of the leaflet, or towards an annulus of the leaflet.

The chosen location of implantation of the leaflet anchor may depend on a number of factors, and may be patient-specific. Implanting the leaflet anchor system in an atrial side of the leaflet may provide a surgeon with greater flexibility in choosing where to implant the leaflet anchor, i.e. by being able to implant the leaflet anchor towards an edge of the leaflet, towards an annulus of the leaflet, or in between.

The tissue of the leaflet closer to the annulus, rather than towards an edge of the leaflet, may be less prone to experiencing trauma associated with the implantation of the leaflet anchor. The tissue towards the annulus of the leaflet may be thicker than that closer to the leaflet edge, for example. The tissue towards the annulus of the leaflet may be more able to withstand the tension associated with the artificial chordae line during the cardiac cycle, when the line is taut.

By implanting the leaflet anchor system from the atrial side, the leaflet anchor system may be able to be implanted closer towards an annulus of the leaflet of the heart whilst still providing adequate support to the edge of the leaflet.

When the artificial chordae line is used to prevent leaflet regurgitation (i.e. mitral regurgitation or tricuspid regurgitation) the line will generally be fixed at two ends, with one end located at/in the papillary muscle of the heart, and the other located at the leaflet anchor system. If the leaflet anchor system is therefore implanted in the leaflet from the ventricular side, the line will extend to the papillary muscle without providing any support to the edge of the leaflet, i.e. at a flailing end of the leaflet. However, when implanted in the leaflet from the atrial side, the line may extend along an atrial-side surface of the leaflet, and extend over the edge of the leaflet before descending into the ventricle to the location of implantation in the papillary muscle. As such, the line may provide support to the flailing edge of the leaflet with the anchor implanted towards the leaflet annulus. This may also better replicate the action of chordae tendineae located towards the edge of the leaflet of the heart valve.

The leaflet anchor system may be arranged to be deployed such that the artificial chordae line will be in contact with an atrial side of the leaflet of the heart between the leaflet anchor and an edge of the leaflet of the heart valve.

When the leaflet anchor system is implanted from the atrial side, it will be understood that the artificial chordae line may provide support to the flailing edge when implanted in the atrial side of the leaflet, given that the line will descend from the atrial side to the ventricular side through the leaflet valve over an edge of the leaflet, when the line is implanted. This may be particularly beneficial when treating flailing leaflets.

The previous catheter devices of WO2016/042022 and WO2020/109596 may generally require precise implantation of the leaflet anchor system in the leaflet to provide adequate support to the edge of the leaflet. As the leaflet anchor system does not provide any additional support to the edge of the leaflet other than its own implantation, the location of implantation of the anchor determines how much support is provided to the edge of the leaflet. Accordingly, implantation of the leaflet anchor system may need to be more precise to ensure adequate support to the edge of the leaflet.

However, due to the contact of the artificial chordae line with the edge of the leaflet when the leaflet anchor system is implanted in the atrial side of the leaflet, the location of implantation of the leaflet anchor system of the present invention need not be so precise, as additional support is provided to the edge of the leaflet regardless of whether the leaflet anchor system is implanted towards the edge of the leaflet or towards the atrial annulus of the leaflet. This may result in more efficient implantation of the leaflet anchor system, as movement of the leaflet during the cardiac cycle which may alter the location of implantation of the leaflet anchor will be of lesser detriment to the overall support provided by the leaflet anchor.

The aforementioned anchor system tube may be a leaflet anchor system tube. The opening of the leaflet anchor system tube may be arranged to be in contact with the atrial side of the leaflet of the heart during deployment.

Placing the opening of the leaflet anchor system tube in contact with the atrial side of the leaflet of the heart during deployment may facilitate the implantation of the leaflet anchor system from the atrial side of the leaflet. For example, placing the opening in contact with the atrial side may ensure proper placement, location and deployment of the anchor system.

The leaflet anchor system may be arranged to be pushed out of the leaflet anchor system deployment mechanism from the proximal end of the catheter device toward the distal end of the catheter device.

The catheter device may comprise the aforementioned linear-shaped rod for deployment of the leaflet anchor system. The linear-shaped rod may be configured to push the leaflet anchor system out of the leaflet anchor system deployment mechanism. The linear-shaped rod may be the wire guide member(s).

The catheter device may comprise a two-part housing section extending from a proximal end of the catheter device along the length of the catheter device toward the distal end of the catheter device. The aforementioned housing section may be the two-part housing section. The two-part housing section may comprise a proximal part at the proximal end of the catheter device and a distal part located on the distal side of the proximal part. The gripper device, the gripper control member and a first anchor system deployment mechanism may be located in the proximal part of the two-part housing section, wherein the first anchor system deployment mechanism is the anchor system deployment mechanism; and the distal part of the two-part housing section may comprise a second anchor system deployment mechanism configured to deploy a second anchor system into further soft body tissue by moving the second anchor system outward in the distal direction relative to the distal part.

The two-part housing section may be arranged to be coincidentally placed between papillary muscle and a leaflet of the heart during use of the catheter device.

The gripper arm may be provided in the proximal part of the two-part housing section and may be rotatably coupled to the catheter device. The gripper arm may be rotatably coupled via any of the above-discussed mechanisms.

The two-part housing section may be formed from two tubular sections in any suitable material, i.e. a medically appropriate material. Stainless steel or nitinol may be used. In the alternative, composite materials such as carbon-fibre or glass-fibre reinforced PEEK may be used. The catheter device may be formed via a combination of such materials with the materials for different parts of the device being selected dependent on the required characteristics of those parts. A material that allows Ultrasound to pass through and at the same time have sufficient strength is preferred, Carbon reinforced PEEK meets these demands well, and would also allow injection moulding of the components which lowers manufacturing cost. Fibre reinforced plastic are normally not visible on X-ray, so strategically placed radiopaque markers in all components may be used to determine device component(s) position and orientation on X-ray relative to each other, as complementary information to ultrasound imaging.

A flexible joint may be located between the proximal part and the distal part of the two-part housing section. The flexible joint allows a centreline of the distal part to be angled relative to a centreline of the proximal part.

The flexible joint may include a hinge element, for example with the distal part of the two-part housing section coupled to the proximal part via a pivoting mechanism or via an elastically deformable element. For example, the two parts of the housing section may be composite or metal parts coupled together by the hinge element.

The first anchor system may be a leaflet anchor system, wherein the soft body tissue is a heart valve leaflet, and the second anchor system may be a papillary anchor system, and wherein the further soft body tissue is papillary muscle. The first anchor system deployment mechanism may thus be a leaflet anchor system deployment mechanism, and the second anchor system may thus be a papillary anchor system deployment mechanism. The catheter device may be for repair of the heart by implanting the leaflet anchor system and the papillary anchor system to secure an artificial chordae line. The leaflet anchor system may be any of the aforementioned soft tissue anchor systems or leaflet anchor systems.

The papillary anchor system may be housed within the distal part of the housing section before its deployment. The papillary anchor system may have a similar cross-section as the distal part of the housing section. For example, both may have a tubular form when the anchor system is held in the distal part. As noted above the anchor system may comprise an anchor having a folded and an unfolded configuration allowing pins of the anchor to form into hooks within the body tissue during deployment of the papillary anchor. The papillary anchor system deployment mechanism may take a similar form to that of WO2016/042022 or WO2020/109596.

In one example the papillary anchor system deployment mechanism includes a first wire or rod for pushing the papillary anchor in the distal direction relative to the distal part of the two-part housing section. There may additionally be a second wire or rod for releasing the papillary anchor from the papillary anchor deployment mechanism in order to disengage the papillary anchor from the catheter device after it is implanted in the body tissue, i.e. the tissue of the papillary muscle and/or tissue adjacent to the papillary muscle.

The papillary anchor may have a chordae line attached to it, and may include a locking mechanism, such as a locking ring as in WO2016/042022 or in WO2020/109596, the locking mechanism being for clamping the chordae line when no force is applied to the locking mechanism. The locking ring may be able to be elastically deformed to release the line from the locking mechanism for adjustment of the length of the chordae line. The papillary anchor system deployment mechanism may include a locking ring holder for holding the locking ring in its elastically deformed position, with the papillary anchor system deployment mechanism being arranged to selectively withdraw the locking ring holder from the locking ring so that the chordae line can be locked in place after deployment of the papillary anchor and after any required adjustment of the length of the chordae line.

Viewed from a second aspect of the present invention, there is provided a method of use of the catheter device of the first aspect for repair of soft body tissue, the method comprising: moving the gripper device from the closed configuration to the opened configuration; bringing a surface of the gripper device into contact with the soft body tissue; moving the gripper device from the opened configuration to the closed configuration so as to grasp the soft body tissue; engaging the gripper lock mechanism; and deploying the anchor system into the soft body tissue.

The method may comprise moving the gripper arm from the closed configuration to the opened configuration; bringing a surface of the gripper arm into contact with the soft body tissue; and moving the gripper arm from the opened configuration to the closed configuration so as to grasp the soft body tissue between the gripper arm and the housing section.

The method of the second aspect may have one or more features corresponding to those of the catheter device of the first aspect. Thus, the above-mentioned description of the catheter device of the first aspect, including but not limited to all technical advantages and alternative embodiments, may be equally applicable to the method of the second aspect.

Certain example embodiments of the invention will now be described by way of example only and with reference to the accompanying drawings, in which:

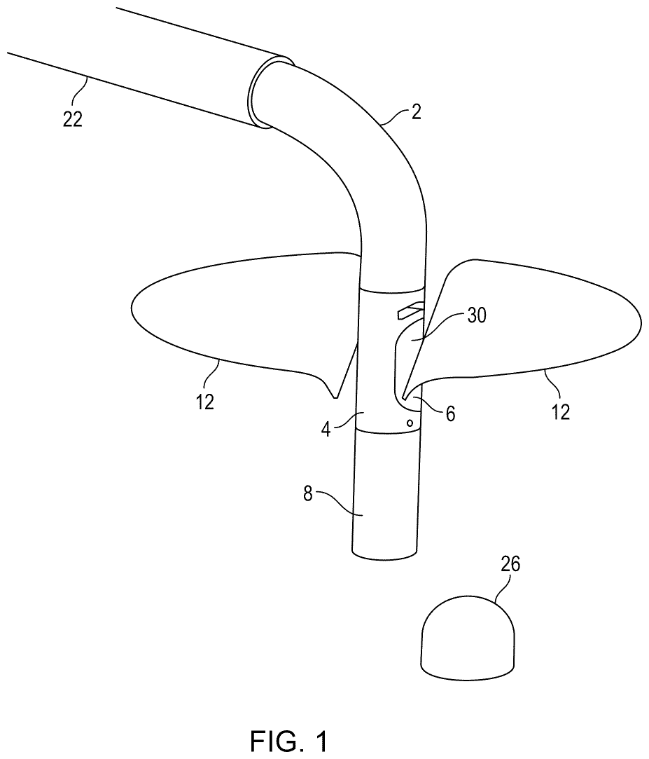

FIG. 1 illustrates the procedure for insertion of a catheter device through a mitral valve;

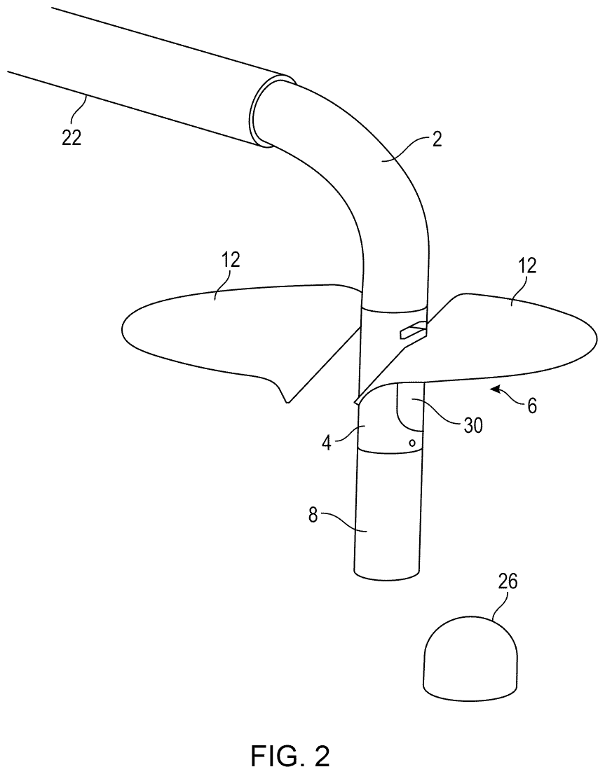

FIG. 2 illustrates gripping of a leaflet of the mitral valve with one gripper arm;

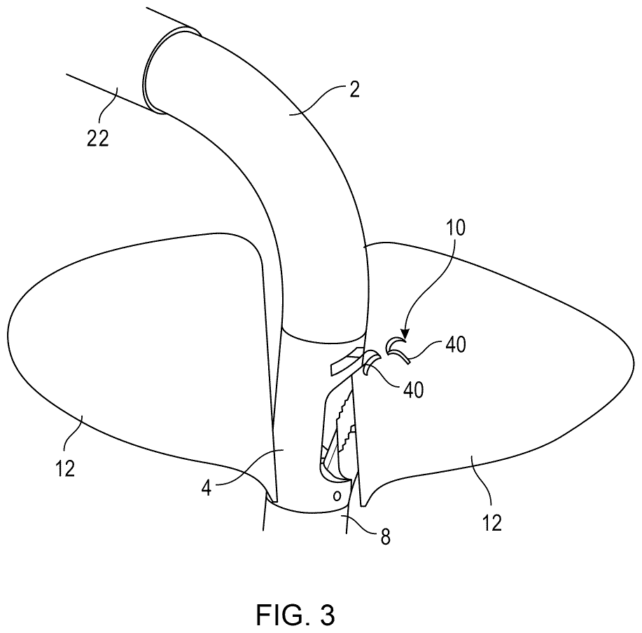

FIG. 3 shows a close up view of the valve during placement of a leaflet anchor, which is coupled to an artificial chordae line;

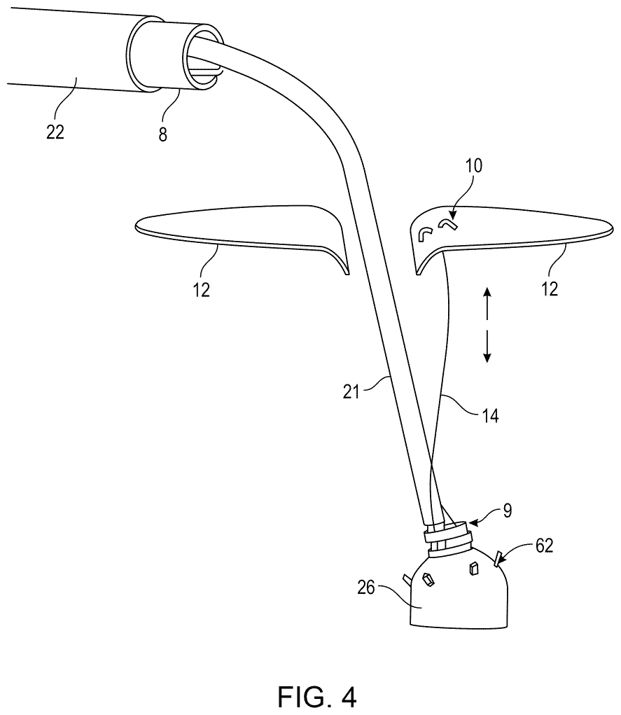

FIG. 4 illustrates withdrawal of a treatment catheter part of the device and adjustment of the chord length with an optional adjustment catheter;

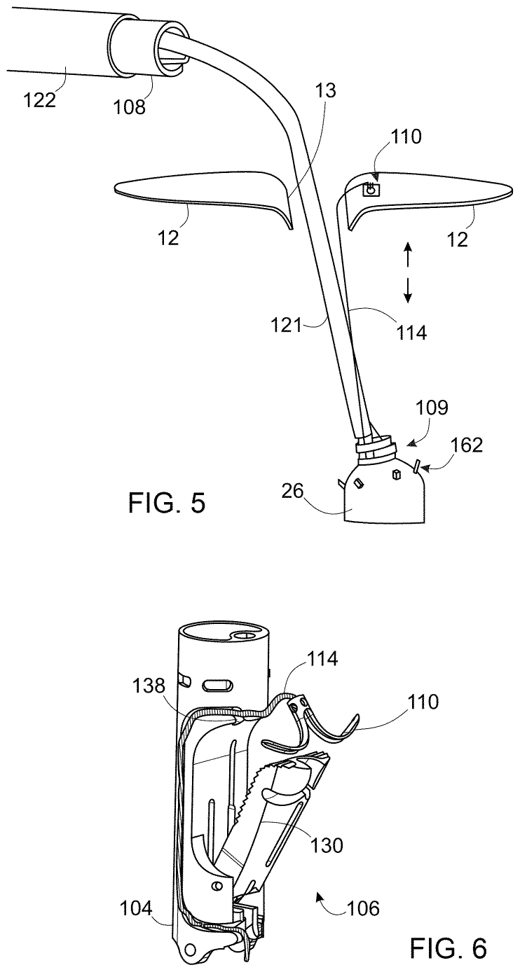

FIG. 5 illustrates withdrawal of a catheter device following implantation of a leaflet anchor in an atrial surface of a leaflet of a heart valve;

FIG. 6 shows the catheter device arranged to implant the leaflet anchor in the atrial surface of the leaflet of the heart valve;

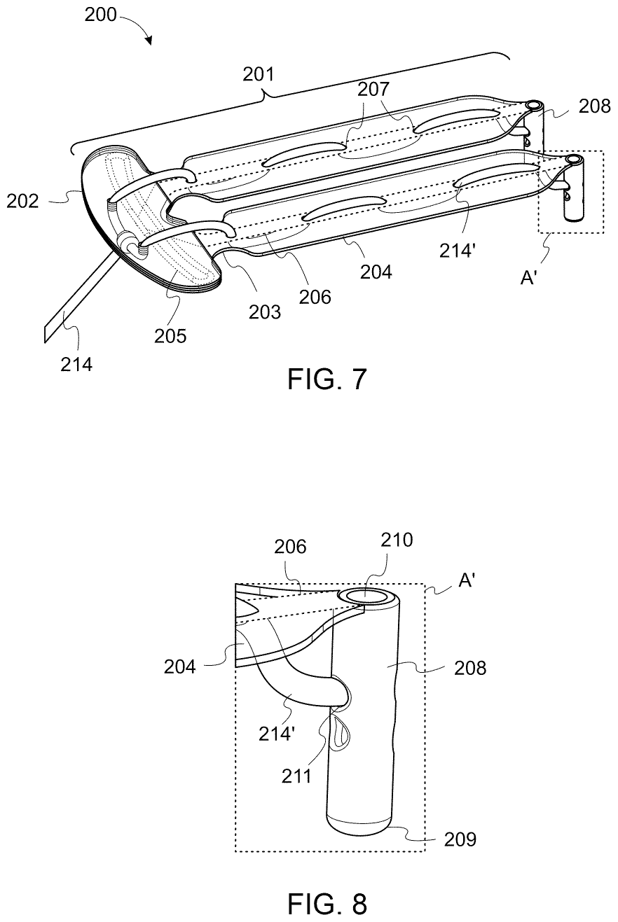

FIG. 7 shows a fabric body anchor system;

FIG. 8 shows a detail view of an end cap of a fabric body anchor system;

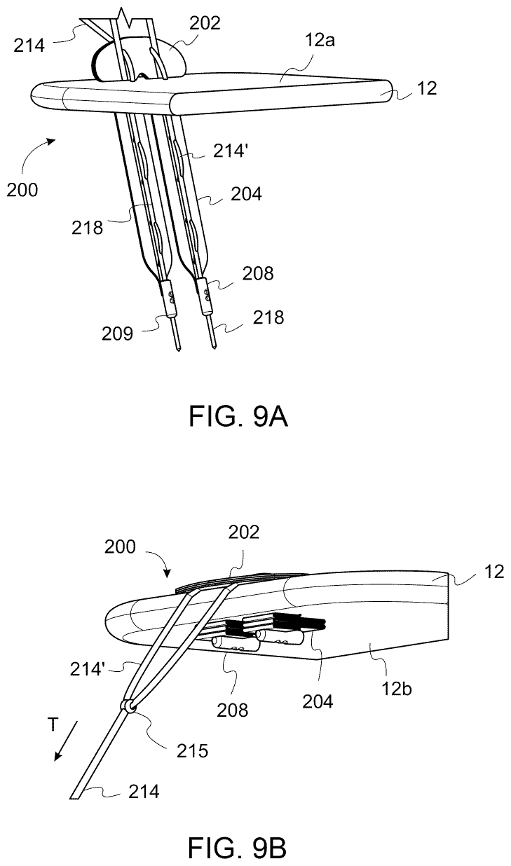

FIG. 9A shows a fabric body anchor system during implantation in a mitral valve leaflet;

FIG. 9B shows a fabric body anchor system following implantation in a mitral valve leaflet from a ventricular-side view;

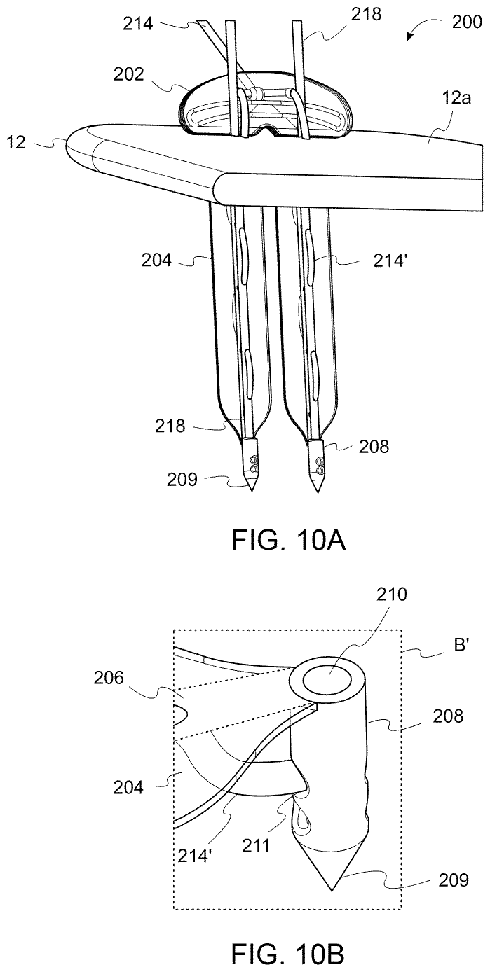

FIG. 10A shows an alternative end cap arrangement of a fabric body anchor system during implantation in a mitral valve leaflet;

FIG. 10B shows a detail view of the end cap arrangement of the fabric body anchor system illustrated in FIG. 10A;

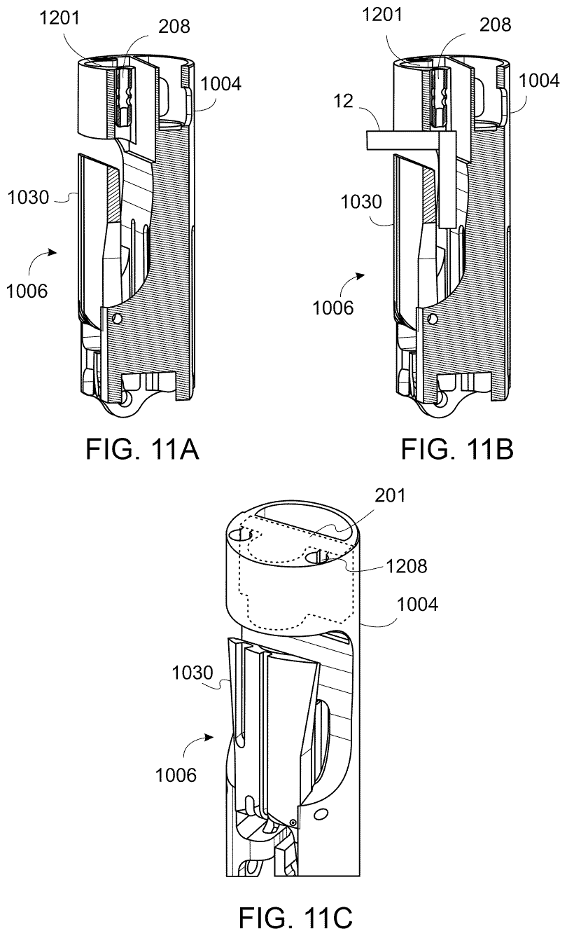

FIGS. 11A to 11C show various schematic illustrations of a soft tissue anchor system housed within a delivery shaft of a catheter device;

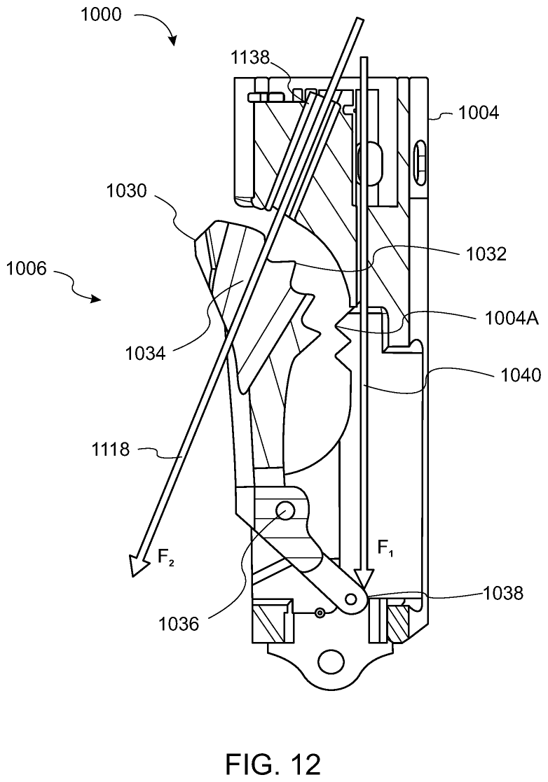

FIG. 12 shows a cross-sectional view of a proximal part of a housing of a catheter device;

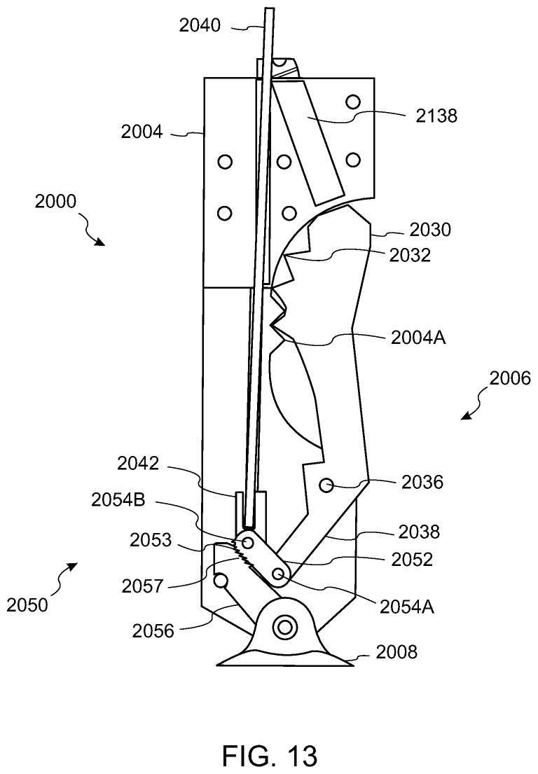

FIG. 13 shows a gripper housing of a catheter device in a cross-sectional view;

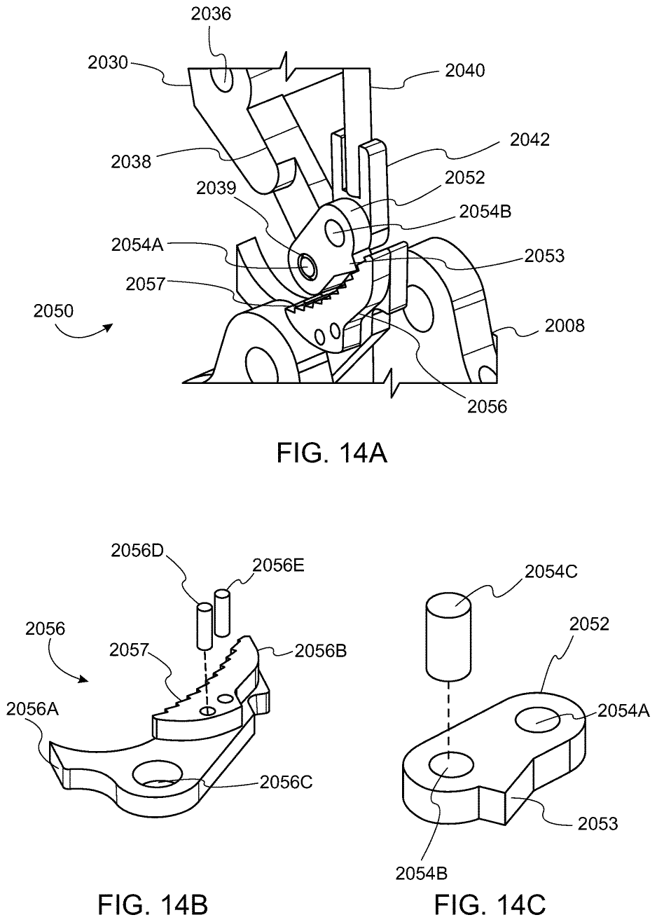

FIG. 14A shows a gripper lock mechanism in a perspective view;

FIGS. 14B and 14C show components of a gripper lock mechanism in an exploded view;

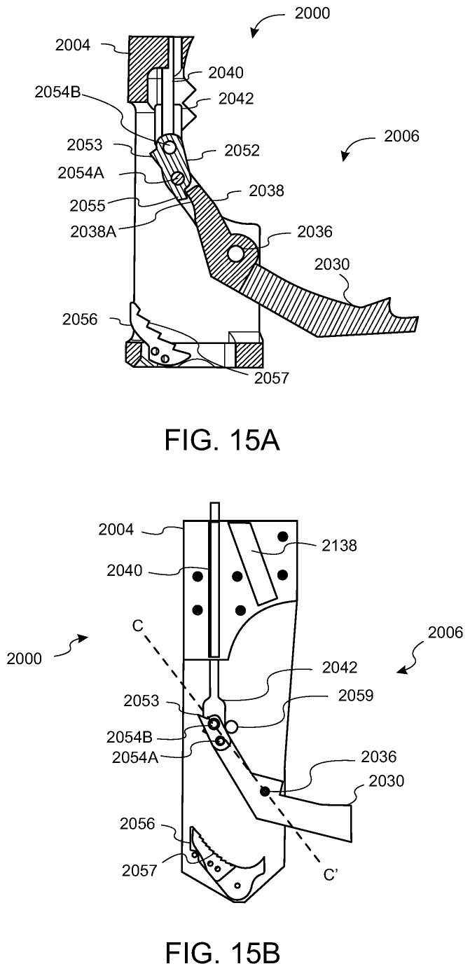

FIGS. 15A and 15B show modified versions of the gripper lock mechanism of FIG. 14 in a cross-sectional view;

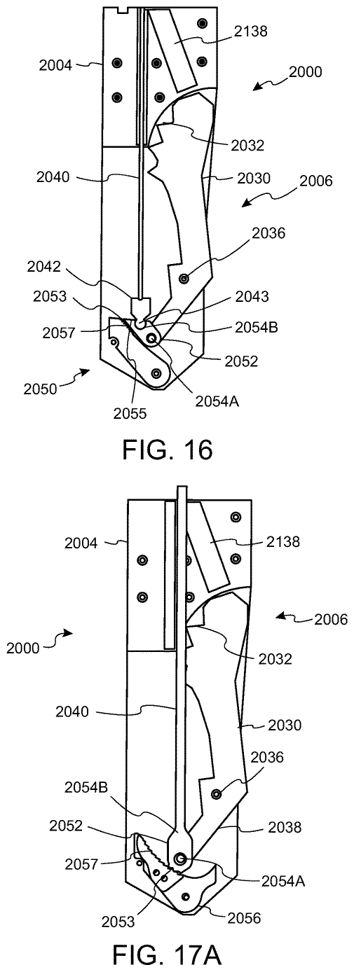

FIG. 16 shows a cross-sectional view of an alternative gripper lock mechanism of a catheter device;

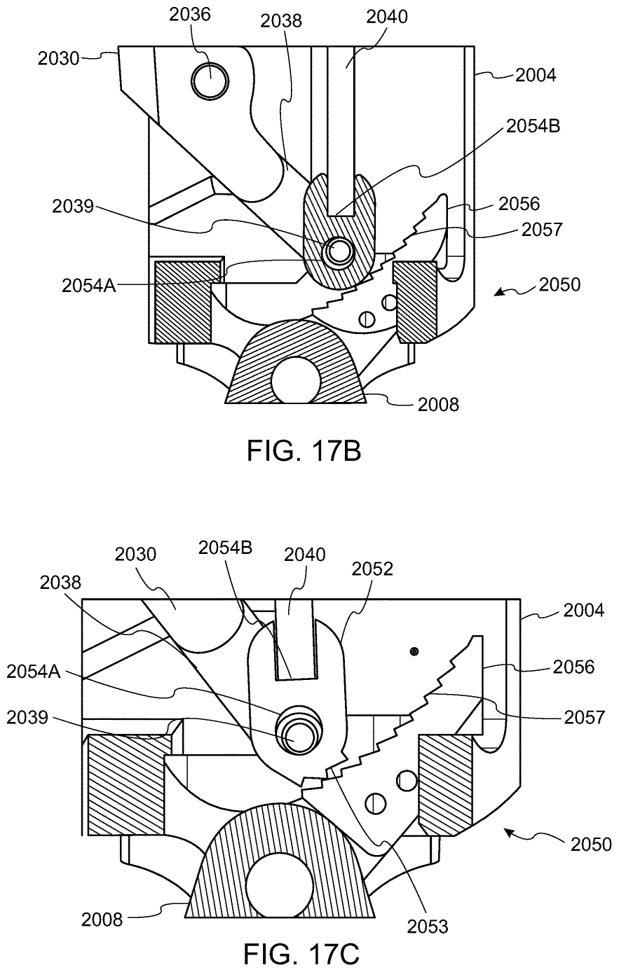

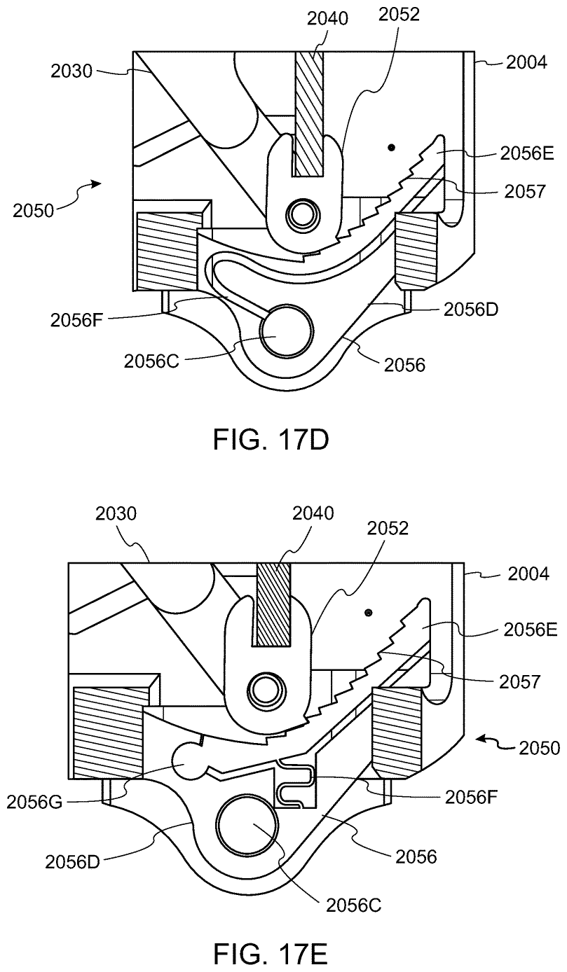

FIG. 17A shows a cross-sectional view of an alternative gripper lock mechanism of a catheter device;

FIGS. 17B to 17E show modified arrangements of the gripper lock mechanism of FIG. 17A in closer detail;

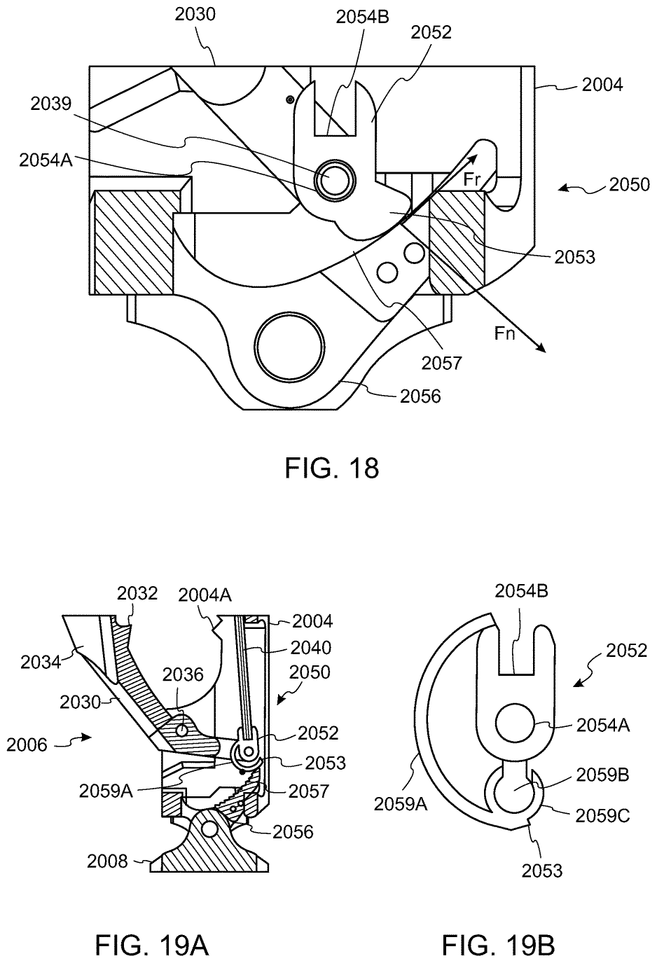

FIG. 18 shows a cross-sectional view of an alternative gripper lock mechanism of a catheter device;

FIG. 19A shows a cross-sectional view of an alternative gripper lock mechanism of a catheter device; and

FIG. 19B shows a modified link member of the gripper lock mechanism of FIG. 20A.

The following description details one or more features consistent with, and combinable with, the aforementioned description of the catheter device of the present invention. The following embodiments herein discussed are not to be viewed in isolation and are not intended to be restrictive, but are to be viewed in the context of the present disclosure as a whole, also considering the appended figures.

The catheter devices presented here are proposed for non-surgical (endovascular) insertion of mitral chords to address mitral regurgitation caused by prolapse of a leaflet 12 of the valve. The figures show different forms of catheter device 2 for this purpose, but it will be understood that the general principles are the same for each device in terms of implantation of a leaflet anchor 10 and a papillary anchor 9 in order to insert one or more artificial chordae lines 14 into the heart. The artificial chordae line(s) 14 are fixed to the prolapsing leaflet 12 and to the papillary muscle 26, thereby recreating a normal anatomy. A single catheter device 2 is used to place both a leaflet anchor 10 and a papillary anchor 9. The length of the chord 14 can be adjusted, again using the same catheter device 2, to eliminate the mitral regurgitation. Thus, such a catheter device enables a single minimally invasive endovascular procedure to be used to repair the mitral valve, providing significant advantages compared to earlier systems requiring more invasive procedures and/or multiple operations.

It should be noted that although an endovascular approach is preferred and the device is hence capable of using this approach, the device could of course be used in different procedures, including more invasive procedures. Many of the advantages will remain, and it could be beneficial to use this device in situations where a more invasive procedure is merited. In addition, it is contemplated that, as discussed above, the catheter device of the present invention can be used for purposes other than the treatment of mitral valve regurgitation, and this disclosure is not intended to be limited in this regard.

The catheter device 2 described in the following can be used to insert mitral chords through the venous system, starting in the femoral vein in the groin. A catheter is advanced to the right atrium. Approach to the left atrium is then gained by a so-called transseptal puncture whereafter a larger guidance catheter is advanced into the left atrium. The catheter device 2 for the heart repair is then introduced through the guiding catheter and into the left atrium.

X-ray and ultrasound guidance is used to position the device and, as explained in more detail below, the mitral leaflet 12 is grabbed and an artificial chordae line 14 is attached using a leaflet anchor 10. The artificial chordae line 14 is then attached to the papillary muscle 26, using a papillary anchor 9. The chord length can now be adjusted to eliminate any mitral regurgitation. Excess chord is then cut and all catheters are withdrawn. Echo and Doppler imaging is used to perform the procedure and monitor the result. The successful use of this endovascular technique will drastically reduce the invasiveness, complications and cost of mitral valve repair.

More detail on the structure and function of the device is set out below with reference to the figures. The procedure of using one form of the device can be summarised as follows:
1) The femoral vein is entered using standard Seldinger technique and the guiding catheter introduced.
2) The guiding catheter is advanced to the right atrium under x-ray guidance.
3) The left atrium is entered after penetration of the atrial septum, guided by x-ray and transesophageal echo.
4) Correct position of the entrance site in the left atrium is verified to assure proper alignment for insertion of the guiding and treatment catheters. The entrance hole in the atrial septum is dilated and the guiding catheter is advanced into the left atrium.
5) A treatment catheter device 2 is advanced through the guiding catheter and positioned in the left atrium above the mitral valve.
6) The prolapsing segment of the mitral leaflet 12 is located with ultrasound and the treatment catheter device 2 is advanced into the left ventricle and a gripper 6 of the treatment catheter device 2 is placed in position to grip the prolapsing segment.
7) The prolapsing segment is gripped and after assuring correct position the leaflet anchor 10 is implanted in and secured to the leaflet 12.
8) The connection of the leaflet to the anchor may be tested whilst it remains attached to the catheter device 2, and if the connection is sufficient then the distal end of catheter is advanced further into the left ventricle.
9) The papillary anchor 9 is pushed into the papillary muscle 26 area and out of its housing 8 thereby letting the papillary anchor 9 open inside the papillary muscle 26.
10) If the gripper 6 is still grasping the leaflet 12 then it is released.
11) The length of the artificial chordae line 14 is adjusted until mitral regurgitation is eliminated.
12) The catheter device 2 is pulled back from the papillary anchor 9, and elimination of mitral regurgitation is again confirmed by echocardiography.
13) The position of the artificial chordae line 14 is locked at the papillary anchor 9.
14) The excess chordae line 14 is cut.
15) Additional artificial chordae lines may be placed if necessary.
16) The catheter device is fully withdrawn and removed from the vascular system.

FIGS. 1 to 6 display an exemplary catheter device 2 as disclosed by WO2020/109596. Whilst the catheter device 2 disclosed in WO2020/109596 is used to implant a leaflet anchor 9 in combination with an artificial chordae line 14 from a ventricular side of a mitral valve leaflet 12, many of the features and/or components of the exemplary catheter device 12 may be compatible with the catheter device 102 of the present invention, or may be modified in accordance with the teachings of the present invention such that a leaflet anchor 110 in combination with an artificial chordae line 114 can be implanted in a leaflet 12 from an atrial side of the leaflet 12, as shown in FIGS. 5 and 6.

FIG. 1 shows guide catheter 22 that has been used to steer a catheter device 2 to a required position within the heart adjacent extending through the mitral valve and hence being between two leaflets 12. The catheter device 2 is composed of four different main parts; a steerable catheter, a gripper housing 4, a gripper device 6 and a papillary anchor housing 8, which holds a papillary anchor 9. The gripper housing 4 and the papillary anchor housing 8 may form a proximal part 4 and a distal part 8 of a two part housing section. The steerable catheter could be replaced with an alternative arrangement using a steerable sheath about a steerable catheter and flexible tubing within the steerable catheter.

FIG. 1 shows a front view of one example catheter device with the gripper device 6 closed. The gripper device 6 of some arrangements uses a single gripper arm 30 that grips the leaflet 12 against the gripper housing part 4. The gripper device 6 is a part of a leaflet anchor deployment mechanism for deploying the leaflet anchor 10 to attach it to the leaflet 12 of the heart. In the example of the figures the gripper device 6 includes a leaflet anchor tube for housing the leaflet anchor 10 prior to deployment. When the gripper device 6 grasps the leaflet 12, the leaflet anchor 10 can be pushed out of the leaflet anchor tube to pierce the leaflet 12 and secure the leaflet anchor 10 in the leaflet 12.

The leaflet anchor 10 is connected to an artificial chordae line 14. The artificial chordae line 14 goes into the papillary anchor housing 8 and through a papillary anchor locking section 28, through a locking and cutting piece. The artificial chordae line 14 can be attached to a wire which passes back along the catheter all the way to the outside (to make the adjustment smoother). The wire allows for a shortening of the chord during the procedure, by pulling, or a lengthening of the chord, since the wire can be pushed through the catheter.

The two-part housing section, with the gripper housing (proximal part) 4 and papillary anchor housing (distal part) 8 might be approximately 6-7 mm in diameter, and approximately 30 mm in length.

FIG. 2 shows a form of gripper mechanism 6 that grasps the leaflet 12 with a single gripper arm that holds it against the gripper housing 4.

A ridged surface on the gripper arm 30 may be provided to help it grip the leaflet 12. 3D ultrasound and/or other available sources can be used to confirm that the gripper mechanism 6 has grasped the correct part of the leaflet 12.

The gripper mechanism 6 can be opened and closed as many times as needed to grasp the right part of the leaflet 12. The opening and closing may be facilitated by a system allowing for one wire to pull the gripper mechanism 6 open, and one to pull it closed. Once the position of the gripper mechanism 6 is confirmed then the leaflet anchor 10 can be pushed out of the end of the leaflet anchor tube, such as by pulling a wire in the other end of the catheter. FIG. 3 shows a close up view of the leaflet anchor 10 placed in the leaflet 12.

If the physician is not satisfied by the connection during the testing (for example, if there is too much movement of the anchor 10 and/or not enough resistance to force on the line) then the leaflet anchor 10 can be retracted and placed in another location.

With the leaflet anchor 10 implanted in the leaflet 12, the papillary anchor housing 8 at the end of the treatment catheter is then placed onto the papillary muscle 26 (not illustrated).

When the distal end of the distal part 8 meets the body tissue, and as further force is applied the counterforce from the body tissue eventually surpasses the forces holding the papillary anchor 9 in place, at this point tissue is pushed flat below the base of the distal part 8 giving a maximal chance of placing all pins 62 of the papillary anchor 9 correctly in tissue, and force can be applied to the papillary anchor 9 so that the ends of the pins 62 then move beyond the distal end of the distal part 8 to meet the body tissue. This may be done via additional force on the papillary anchor 9 from rods or wires 60 or extending the adjustment catheter 21, or it may be done through a pre-tension on the papillary anchor 9 (or friction between the adjustment catheter 21 and the distal part 8) that is held by friction with the distal part until the forces from the body tissue on the distal part 8 changes the balance of forces with the friction sufficiently so that the papillary anchor 9 ejects in a way similar to a paper stapler. As the papillary anchor 9 is ejected the pins 62 fold out and form into the hook shape of the unconstrained papillary anchor 9 to thereby engage with the body tissue 26. At this point the connection can be pull tested by operator, and/or visually confirmed on x-ray and/or ultrasound. If the connection is not satisfactory, the papillary anchor 9 can be pulled back into the distal part 8 and re-placed to attempt an improved coupling of the anchor 9 with the body tissue 26.

FIG. 4 shows the possible next steps. The main part 4, 8 of the device is retracted to minimize influence on the moving leaflets 12. An adjustment catheter 21 can remain at the papillary anchor 9. The length of the artificial chordae line 14 can be adjusted with a wire from the outside. The length is continuously adjusted and the functioning of the leaflet 12 is monitored. The length of the artificial chordae line 14 can be reduced by pulling the chord wire back through the catheter. The length can also be increased by pushing the chord wire, which will slacken the artificial chordae line 14 and allow the movement of the leaflet 12 to pull it out of the adjustment catheter 21. The small size of the adjustment catheter 21 means that the effect of the device on the functioning of the leaflet 12 is minimised. The right length for the artificial chordae line 14 is confirmed with 3D ultrasound and/or other available sources.

When the correct length is confirmed then the device is disengaged from the papillary anchor 9. This process also locks the artificial chordae line 14 in place and cuts off any excess, which is retained in the catheter and withdrawn from the body when the catheter is removed. A locking segment 28 of the papillary anchor 9 is held open by the cutting piece (not shown). The locking segment 28 is a band of the papillary anchor 9 that can be flexed to open a gap for the artificial chordae line 14 to pass through. In the natural shape of the papillary anchor 9, when no force is applied, this locking segment 28 fits closely with the remainder of the anchor 9 and so it will hold the artificial chordae line 14 in place. The locking segment 28 is held open until the artificial chordae line 14 is the correct length. The cutting piece cuts the artificial chordae line 14, which is pulled against the blade when the adjustment process is completed.

The catheter device 2 disclosed in each of WO2016/042022 and WO2020/109596 implants the leaflet anchor 10 from a ventricular side of the leaflet 12. As shown in FIG. 4, the artificial chordae line 14 therefore descends from the leaflet 12 from a ventricular surface of the leaflet 12 to the papillary muscle 26. A number of benefits are associated with implanting the leaflet anchor 10 or the soft tissue anchor system discussed below, and hence the artificial chordae line 14, in a ventricular side of the leaflet 12, as discussed in each of WO2016/042022 and WO2020/109596.

However, there may be situations in which it is advantageous to implant a leaflet anchor 110, and hence an artificial chordae line 114, from an atrial side of the leaflet 12. For example, as can be seen in FIG. 4 the artificial chordae line 14 descends to the papillary muscle 26 without providing any additional support to an edge 13 of the leaflet 12. When implanted from a ventricular side of the leaflet 12, the artificial chordae line 14 does not provide additional support to the edge 13 of the leaflet 12. The implanted artificial chordae line 14 may therefore not replicate the action of chordae tendineae located towards the edge 13 of the leaflet 12 as accurately as desired.

Implanting the leaflet anchor 10 from a ventricular side of the leaflet 12 also requires a more precise placement of the leaflet anchor 10. As there is no additional support provided to the edge 13 of the leaflet 12, the placement of the leaflet anchor 10 will determine to what extent the edge 13 of the leaflet 12 is supported and/or secured by the leaflet anchor 12. In contrast, the placement of a leaflet anchor 110 implanted from the atrial side of the leaflet 12 can be less precise, since the artificial chordae line 114 will provide additional support to the edge 13 of the leaflet 12 as it passes into the ventricle from the atrium of the heart.

FIG. 5 shows the withdrawal of a guide catheter 122 and a distal part 108 of a catheter device 102 once an artificial chordae line 114 has been implanted in the papillary muscle 26 using a papillary anchor 109, and once the artificial chordae line 114 has also been implanted in the leaflet 12 of the heart using a leaflet anchor 110. The adjustment catheter 121 is shown in place before its withdrawal. The length of the artificial chordae line 114 can be adjusted as necessary in the illustrated configuration.

FIG. 5 is similar to the arrangement shown in FIG. 4, but shows the leaflet anchor 110 implanted from an atrial side of the leaflet 12 rather than a ventricular side. As can be seen in FIG. 2, the artificial chordae line 114 extends from a base of the leaflet anchor 110 where it is attached towards the leaflet edge 13. As the artificial chordae line 114, when under tension or otherwise, will take the shortest possible path to the papillary muscle 26 where the other end is implanted, the artificial chordae line 114 will be in contact with the atrial side surface of the leaflet 12 and will descend, in contact, over the edge 13 of the leaflet 12. As such, the artificial chordae line 114, implanted in an atrial side of the leaflet 12, will provide additional support to the edge 13 of the leaflet 12.

The artificial chordae line 114 can comprise regions of varying cross-sectional area along its length. By increasing the cross-sectional area of the artificial chordae line 114 in certain sections, the artificial chordae line 114 can have an increased area of contact with the leaflet 12 of the heart. As such the force applied by the artificial chordae line 114 to the leaflet 12 may be more evenly distributed, and any pinching of the leaflet 12 which the artificial chordae line 114 may cause can be avoided.

The artificial chordae line 114 comprises a flattened cross-section proximal to the leaflet 12, i.e. such that a major axis of the cross-sectional area of the artificial chordae line 114 lies parallel to the surface of the leaflet 12. In alternative arrangements, the artificial chordae line 114 can be formed of a plurality of sutures, such that an area of contact between the artificial chordae line 114 and the atrial surface of the leaflet 12 is increased.

To implant the leaflet anchor 110 in the leaflet 12 of the heart from an atrial side, a leaflet anchor deployment mechanism and a gripper housing 106 of the catheter device 102 is arranged as shown in FIG. 6.

FIG. 6 shows the catheter device 102 comprising a gripper housing 106, a gripper arm 130 and the artificial chordae line 114 attached to the leaflet anchor 110 or the soft tissue anchor system discussed below and routed through the main body of the catheter device 102. In the example of these figures the leaflet anchor 110 is housed in the main body of the catheter device 102, and is deployed by pushing it out of a leaflet anchor tube 138 located in the main body of the catheter device 102. The leaflet anchor tube 138 is similar in function to the leaflet anchor tube described above. The leaflet anchor tube 138 is located in the main body of the catheter device 102 such that when the leaflet 12 is grasped between the gripper arm 130 and the main body of the catheter device 102, the leaflet anchor 110 can be deployed from the anchor tube 138 into an atrial side of the leaflet 12. The leaflet anchor 110 will hence be deployed in the leaflet 12 as shown in FIG. 5.

In the arrangement shown in FIG. 6, the leaflet anchor tube 138 extends in a direction along the main body of the catheter device 102 or the gripper arm 130 such that the opening of the leaflet anchor tube 138 opens towards a distal end of the catheter device 102. As the catheter device 102 approaches the leaflet 12 and the papillary muscle 26 from above the leaflet 12 and the papillary muscle, i.e. from the left atrium as discussed above, the opening of the leaflet anchor tube 138 is therefore arranged to meet the atrial surface of the leaflet 12, such that the leaflet anchor 110 can be implanted in the atrial surface of the leaflet 12.

The catheter devices taught in each of WO2016/042022 and WO2020/109596 used a U-rod to deploy the leaflet anchor. However, the catheter device 102 employs a linear rod to deploy the leaflet anchor 110. The linear rod will extend from a proximal end of the catheter device 102 and into the leaflet anchor tube 138, such that the leaflet anchor 110 can be deployed into the atrial side of the leaflet 12. A linear rod may deploy an anchor system by pushing it out of the distally-facing opening of the leaflet anchor tube 138, using the end of the linear rod located in the leaflet anchor tube 138. The linear rod is flexible so that it can curve or bend, e.g. as it extends into the leaflet anchor tube 138, and is tensile so that it can be pushed into and retracted from the leaflet anchor tube 138 without elongating. The linear rod is made of a material with the ability to deform elastically to a high degree in order to allow for the bending of the bendable section. Suitable materials include shape memory materials, for example shape memory metals such as nitinol. Using a shape memory metal also means that the linear rod can be made to be stiff, which makes the transfer of force with the linear rod more efficient. Alternatively, the linear rod could be made of several types of materials to achieve the required properties.

Whilst the following features will be discussed in relation to the catheter device 102 as discussed in relation to FIGS. 5 and 6, it will be appreciated that the following features are similarly compatible with the catheter device 2 as discussed in relation to FIGS. 1 to 4 and as disclosed in each of WO2016/042022 and WO2020/109596.

In the arrangements illustrated in FIGS. 1 to 6, the leaflet anchor 10, 110 deploys from a folded configuration (e.g. due to its containment in the leaflet anchor tube) to an unfolded configuration. In the unfolded configuration, hooked formations 40 unfold so that the leaflet anchor 10 is secured in the leaflet 12. These hooked formations extending collinearly with the leaflet anchor tube when in the folded configuration, and are used to pierce the leaflet 12 during implantation of the leaflet anchor 10.

A soft tissue anchor system 200 can be used in place of the leaflet anchor 10. The soft tissue anchor system 200 is illustrated in FIGS. 7 to 10.

FIG. 7 illustrates a soft tissue anchor system 200 comprising a U-shaped fabric body 201. The U-shaped fabric body 201 comprises a base portion 202 and two arm portions 204 extending from the base portion 202. A narrow waist portion 203 extends between the base portion 202 and each respective arm portion 204. Soft tissue anchor systems 200 comprising a plurality of arm portions 204 connected by a single base portion 202 may increase a surface area of the anchor system 200 when engaged with body tissue, on both sides of the body tissue. This may improve the stability of the anchor system 200 when implanted in body tissue.

The U-shaped fabric body 201 is used to anchor an artificial chordae line 214, located towards the base portion 202, to body tissue. Being a fabric body, it will be understood that the U-shaped fabric body 201 is primarily formed of a soft material. The U-shaped fabric body 201 may be comparable in function to a pledget. As illustrated in FIG. 7, the base portion 202 comprises a shape retention feature 205 embedded therein, such as a nitinol wire frame or the like. The shape retention feature 205 may help the base portion 202 retain its shape over time and/or provide additional lateral support to the body tissue when implanted. The arm portions 204 can also optionally be provided with stiffening elements 206.

The U-shaped fabric body 201 also comprises a tensile line 214′. Each arm portion 204 comprises a portion of tensile line 214′ threaded through the arm 204 and the base 202, the threading running from an end of the arm portion 204 distal to the base portion 202, to the base portion 102. Each portion of tensile line 214′ is fixed towards the distal ends of the arm portions 204, but is otherwise free to move with respect to the threaded holes 207 formed along the arm portions 204 and in the base portion 202.

Each arm portion 204 is configured to collapse in folds towards the base portion 202, by action of the tensile line 214′ threaded through the arm portions 204. That is, the tensile line 214′ can collapse the arm portions 204 in folds towards the base portion 202 when a tension force is applied to the tensile line 214′. In other words, the tensile line 214′, threaded through the arm portions 204, is an example of a means for collapsing each arm portion 204 in folds towards the base portion 202 such that, in use, the body tissue is sandwiched between the base portion 202 and each of the arm portions 204. As the U-shaped fabric body 201 is formed of a primarily soft material, actuation of the tensile line 214′ from an end distal to where the portions of line 214′ are fixed to the arm portions 204 results in the arm portions 204 collapsing in a concertina fashion (i.e. collapsing in folds towards the base portion 202).

Also located at the ends of the arm portions 204 distal to the base portion 202 are end caps 208. The end caps 208 are rigid structures which aid implantation of the U-shaped fabric body 201 in the body tissue, and also assist in maintaining engagement of the U-shaped fabric body 201 to the body tissue.

FIG. 8 shows detail A′ of FIG. 7 from a closer perspective. The portions of line 214′ threaded through the arm portions 204 are fixed to the distal ends of the arm portions 214′ via the end caps 208. In the embodiment illustrated in FIGS. 7 and 8, each respective end of the tension line 214′ is received by an opening 211 of each respective end cap 208, and is fixed therein. Each end cap 208 also comprises an opening 210 for receiving a wire guide member 218. The use of end caps 208 which receive wire guide members 218 for implanting the anchor system 200 may facilitate ease of implantation, because the fabric body 201 need not be implanted from within a hollow needle. Accordingly, trauma to the site of implantation will be reduced, as a narrower piercing member may be used to deploy the soft tissue anchor system 200.

FIGS. 9A and 9B show the soft tissue anchor system 200 implanted in a mitral valve leaflet 12 of the heart. The base portion 202 is arranged to contact an atrial surface 12a of the leaflet 12, and the concertinaed arm portions 204 (as in FIG. 9B) are arranged to contact a ventricular surface 12b of the leaflet 12. The leaflet 12 is therefore sandwiched between the base portion 202 and the arm portions 204, in use.

As can be seen in FIG. 9B, the artificial chordae line 214 provides a tensile force as indicated by arrow T to the tensile line 214′, thereby drawing the fixed ends thereof towards the base portion 202. This results in the arm portions 204 collapsing in folds into the concertinaed position.

The artificial chordae line 214 can be joined to the tensile line 214′ via any suitable fastening means, for example a knot or an eyelet. In the embodiment shown in FIG. 9B, the artificial line 214 is joined to the tensile line 214′ via a knot 215.

Referring now to FIG. 9A, to implant the U-shaped fabric body 201, a pair of wire guide members 218 are employed. The wire guide members 218 are each received by the opening 210 of the end cap 208. The wire guide members 218 are used to push the end caps 208 into a piercing engagement with the leaflet 12, and through the leaflet 12, such that the U-shaped fabric body 201 engages the leaflet 12. In this configuration, the end caps 208 extend collinearly with the arm portions 204. The wire guide members 218 can then be retracted once the U-shaped fabric body 201 is implanted in the leaflet 12.

The wire guide member 218 illustrated in FIG. 10A passes through the end cap 208 and is itself used to pierce the leaflet 12, whilst simultaneously pushing the end caps 208 through the leaflet 12. In the arrangement shown in FIG. 9A, the wire guide member 218 comprises a piercing section 219. The piercing section 219 comprises a distal tip that is configured to pierce the leaflet 12 during implantation of the soft tissue anchor system 200. Thus, in the arrangement shown in FIG. 9A, the wire guide member 218 may be regarded as a piercing wire guide member 218. The opening 210 of the end cap 208 appreciably extends along the entire length of the end cap 208, to enable passage of the piercing wire guide member 218 therethrough. To aid with manipulation of the end caps 208, the wire guide member 218 comprises a thicker control section 220. A shoulder portion 221 is located between the thinner piercing section 219 and the thicker control section 220, and is configured to engage a complementary portion of the end cap 208. Transmitting a force to the end caps 208 in this manner results in the arm portions 204 being pulled through the implantation site created by the respective piercing sections 219 of the wire guide members 218.

Alternatively, as shown in FIGS. 10A and 10B, the tip 209 of the end cap 208 distal to the arm portions 204 is pointed, such that the end cap 208 is capable of piercing the leaflet 12. Where the tip 209 of the end cap 208 is pointed, the wire guide members 218 are used to transmit a force to the end caps 208 to both pierce the leaflet 12 and to pull the arm portions 204 through the implantation sites created in the leaflet 12 by the pointed tips 209 of the end caps 208.

With the use of the soft tissue anchor system 200 discussed above, the catheter device 102 can be adapted to replace the leaflet anchor tube 138 of FIGS. 1 to 6 with an alternative anchor deployment mechanism, such as tubes of different design for holding and guiding the arm portion(s) of the fabric body as they are implanted into the leaflet.

FIGS. 11A to 11C schematically illustrate how the soft tissue anchor systems 200 of FIGS. 7 to 10 may be housed by, and deployed from, a catheter device. Each of FIGS. 11A to 11C show only a proximal part 1004 of a catheter device, but it will readily be appreciated that such a proximal part 1004 could be implemented in a catheter device as discussed in relation to any of FIGS. 1 to 6.

FIG. 11A shows an end cap 208 of a soft tissue anchor system 200 housed in a channel or groove 1208 for deployment from the proximal part 1004 of a catheter device. The proximal part 1004 functions as a gripper housing 1006, and accordingly the channel 1208 faces the gripper arm 1030 such that when a leaflet 12 is grasped by the gripper arm 1030, as illustrated in FIG. 11B, the end cap 208 can be deployed from the channel 1208 by actuation of a respective wire guide member 218.

The proximal part 1004 of the catheter device also provides a housing 1201 for the U-shaped fabric body 201. FIG. 11C illustrates the U-shaped fabric body 201 when stowed in the housing 1201. The U-shaped fabric body housing 1201 also faces the gripper arm 1030, such that the U-shaped fabric body 201 can be implanted into a grasped leaflet 12.

The U-shaped fabric body 201 is placed in a thin tubular sheath, or curtain, in the housing 1201. The sheath acts to reduce friction experienced by the U-shaped fabric body 201 from the housing 1201 during deployment of the soft tissue anchor system 200 from the catheter device. The sheath can deflect, or crumple, during deployment to aid deployment of the fabric body 201.

The U-shaped fabric body housing 1201 is open to the channels 1208 for the end caps 208, such that the arm portions 204 can extend between the end cap channels 1208 and the U-shaped fabric body housing 1201. This arrangement may accordingly facilitate smooth deployment of the soft tissue anchor system 200 from the proximal part 1004 of the catheter device, into a grasped leaflet 12.

FIG. 12 shows a catheter device 1000, and in particular the proximal part 1004 of the housing of the catheter device 1000. The proximal part 1004 functions as a gripper housing 1006 and also as a housing for an anchor system, such as a leaflet anchor system, deployable from a leaflet anchor system tube 1138. The gripper housing 1006 comprises a gripper arm 1030, which in the illustrated arrangement is a single gripper arm 1030. A wire guide member 1118, i.e. a control wire configured to deploy the leaflet anchor system from the leaflet anchor system tube 1138; and a single gripper control wire 1040, i.e. a control wire configured to actuate a gripper arm 1030 of the gripper housing 1006, are also provided.

The gripper arm 1030 comprises a plurality of serrations 1032. The serrations 1032 increase an area of contact between a leaflet 12 and the gripper arm 1030, when the gripper arm 1030 is used to grasp the leaflet 12 between itself and the proximal part 1004 of the housing of the catheter device 1000. The serrations 1032 face the opening of the leaflet anchor system tube 1138. The proximal part 1004 of the housing also comprises a plurality of serrations 1004A, formed on the surface facing the gripper arm 1030.

The gripper arm 1030 also comprises an internal space 1034. The internal space 1034 is formed in the surface of the gripper arm 1030 facing the leaflet anchor system tube 1138. The internal space 1034 is configured to receive a wire guide member 1118 and the leaflet anchor system during deployment of the leaflet anchor system into the leaflet 12. The internal space 1034 facilitates a complete extension of the wire guide member 1118 during implantation of the anchor system in soft body tissue. As illustrated, the internal space 1034 defines an opening through the gripper arm 1030. In other arrangements, the internal space 1034 can be a cavity formed in the surface of the gripper arm 1030.

The gripper arm 1030 is rotatably coupled to the proximal part 1004 of the housing, and in the present arrangement this is achieved via a hinge 1036. The gripper arm 1030 also comprises a lever portion 1038. The gripper control wire 1040 is configured to actuate the gripper arm 1030 via the lever portion 1038. The gripper control wire 1040 applies a force to the lever portion 1038, which creates a moment about the hinge 1036. The gripper arm 1030 accordingly rotates about the hinge 1036 depending on the control input. In this manner, the gripper arm 1030 is capable of rotating out of, and away from, the proximal part 1004 of the housing of the catheter device 1000.

In use, the gripper arm 1030 is rotated away from the housing by pulling the gripper control wire 1040 (i.e. by withdrawing the gripper control wire 1040 in a proximal direction, or back into a delivery catheter used to deliver the catheter device 1000). The catheter device 1000 is positioned such that the gripper arm 2030 can grasp the leaflet 12. The gripper arm 1030 is used to then grasp the leaflet 12 by pushing the gripper control wire 1040 (i.e. by forcing the gripper control wire 1040 in a distal direction, or out of the delivery catheter and into the catheter device 1000). The leaflet 12 can be firmly and stably grasped by applying a force F₁ via the gripper control wire 1040, to keep the gripper arm 1030 closed.

With the leaflet 12 grasped in place, the leaflet anchor system can then be deployed from the leaflet anchor system tube 1138 by applying a deployment force F₂ via the wire guide member 1118. The deployment force F₂ causes the leaflet anchor system to pierce the leaflet 12, and thereby be implanted in the leaflet 12.

During implantation of the leaflet anchor system in the leaflet 12, the application of deployment force F₂ by the wire guide member 1118 creates a moment about the hinge 1036 that acts to open the gripper arm 1030. This may result in the leaflet 12 being less stably or firmly grasped in the gripper housing 1006. To prevent the gripper arm 1030 from opening during implantation of the leaflet anchor system, a greater force can be applied via the gripper control wire 1040. However, the application of greater force may increase a risk of trauma to the leaflet 12 and the surrounding body tissue, and may also increase a risk of malfunction within the catheter device 1000.

Embodiments of the present invention therefore provide a catheter device comprising a gripper lock mechanism. The gripper lock mechanism is configured to maintain the gripper arm in a closed position when grasping the leaflet 12, particularly during implantation of the anchor system in the leaflet 12 when a deployment force F₂ is applied by the wire guide member 1118. In particular, embodiments of the present invention contemplate an arrangement wherein a gripper control wire is used to control both the position of the gripper arm and the gripper lock mechanism.

The catheter devices described herein may have one or more features of the catheter device 2, 1002 of the type described above in connection with FIGS. 1 to 12, with the gripper housing or device 6, 106, 1006 shown in those figures replaced by the gripper arm described below and discussed in relation to the remaining figures, with other modifications being apparent to the person skilled in the art.

The catheter devices illustrated in, or discussed in relation to, the remaining figures are for implantating an anchor, such as an anchor in combination with a line, in soft body tissue and, more specifically, can be used as a catheter device for use in the surgical repair of mitral valve leaflets, the catheter device being used to attach an artificial line to a grasped leaflet of the heart during the repair. This type of repair is discussed in both WO2016/042022 and WO2020/109588, and is also described above in relation to FIGS. 1 to 12.

FIG. 13 illustrates the proximal part 2004 of a catheter device 2000 according to an embodiment of the present invention. The catheter device 2000 is of a similar form and structure to that of the catheter device 1000 illustrated in FIG. 12, wherein like reference numerals denote alike features.

The catheter device 2000 comprises a gripper lock mechanism 2050. The gripper lock mechanism 2050 is configured to maintain the gripper arm 2030 in a closed position when grasping the leaflet 12, e.g. when the gripper arm 2030 is flush with, in contact with, or pulled against, the proximal part 2004 of the housing. The gripper control wire 2040 is configured to open and close the gripper arm 2030, and is also configured to operate the gripper lock mechanism 2050.

When engaged, the gripper lock mechanism 2050 can provide a constant amount of force resisting opening of the gripper arm 2030. The resistive force is provided in a direction that is perpendicular, or has a major force component substantially perpendicular, to the direction in which the gripper control wire 2040 applies force F₁ to the gripper arm 2040. The resistive force is also provided at a distance away from a centre of rotation of the gripper arm 2040 (e.g. around a hinge 2036 or other suitable pivoting means) so as to provide a greater moment of resistive force opposing the deployment force F₂ applied to the anchor system.

Accordingly, the gripper lock mechanism 2050 may prevent, or significantly resist, the gripper arm 2030 opening during implantation of the leaflet anchor system. For example, for a force F₁ of 10 N applied by the gripper control wire 2040 keeping the gripper arm 2030 shut, the gripper arm 2030 may be prevented from opening for deployment forces F₂ of 37 N or less. This is in contrast to arrangements not comprising the gripper lock mechanism 2050, where deployment forces F₂ of 3 N may otherwise open the gripper arm 1030 when a force F₁ of 10 N is applied by the gripper lock member 1040. Use of the gripper lock mechanism 2050 may therefore provide a marked improvement in the ability of the gripper arm 2030 to avoid inadvertently opening under action of the deployment force F₂.

By using a gripper lock mechanism 2050 to maintain the gripper arm in the closed position, a lower force is required to be applied by the gripper control wire 2040 when grasping the leaflet 12. In other words, the gripper lock mechanism 2050 can increase the load required to open the gripper arm 2030 when otherwise closed, thereby reducing the force required to be provided by the gripper control wire 2040 to resist the gripper arm 2030 inadvertently opening. This may improve the stability of the leaflet 12 in the gripper housing 2006 during deployment of the leaflet anchor system.

Further, providing a gripper control wire 2040 that can be used to both open and close the gripper arm 2030 and to engage the gripper lock mechanism 2050 may keep the design of the catheter device 2 simple and more compact. For example, operating the gripper housing 2006 using just a single control input may prevent malfunctions associated with multiple control inputs occurring. The catheter device 2000 may be made more compact or simple because only a single gripper control wire lumen may be required to provide complete operation of the gripper housing 2006.

Whilst in the present embodiment the gripper control wire 2040 is used to open and close the gripper arm 2030, in other embodiments the gripper control wire 2040 may be replaced by an alternative gripper control member, such as a gripper control rod, gripper control piston or other suitable member for controlling the gripper arm.

In the present embodiment, the gripper lock mechanism 2050 comprises a link member 2052 and a lock member 2056. The link member 2052 comprises a first end 2054A and a second end 2054B. The link member 2052 is rotatably coupled to the gripper arm 2030 at the first end 2054A, and to the gripper control wire 2040 at the second end 2054B. The gripper arm 2030 is rotatably coupled to the link member 2052 towards an end of the lever portion 2038. The gripper control wire 2040 is rotatably coupled to the link member 2052 via a joint member 2042. The joint member 2042 facilitates the connection between the link member 2052 and the gripper control wire 2040. The lock member 2056 is fixed to the proximal part 2004 of the catheter device 2000 within the gripper housing 2006. In the present embodiment, the lock member 2056 is fixed towards a distal part 2008 of the housing of the catheter device 2000.

The link member 2052 comprises a first locking portion 2053 and the lock member 2056 comprises a second locking portion 2057. The first locking portion 2053 is configured to engage the second locking portion 2057 when the gripper lock mechanism 2050 is engaged.

As illustrated in FIG. 13, in the present embodiment the first locking portion 2053 and the second locking portion 2057 each comprise a plurality of complementary teeth. The second locking mechanism 2057 is, or acts as a ratchet gear rack; and the first locking mechanism 2053 is a plurality of teeth or protrusions configured to mate with the ratchet gear rack, such that the link member 2052 functions as a pawl. The gripper lock mechanism 2050 can accordingly be regarded as a ratchet gear rack mechanism.

Since the first and second locking portions 2053, 2057 comprise a plurality of complementary teeth, the gripper arm 2030 is capable of being locked at a plurality of positions. In the present embodiment, the plurality of positions is a plurality of discrete positions. At its most open position, the gripper arm 2030 is a distance of around 2.8 mm from the adjacent surface of the proximal part 2004 of the housing. When closed, the gripper arm 2030 abuts the adjacent surface of the proximal part 2004 of the housing. In such a position, the serrations 2032 of the gripper arm 2030 engage the serrations 2004A of the adjacent surface of the proximal part 2004 of the housing.

By providing a gripper lock mechanism 2050 that enables the gripper arm 2030 to be locked at a plurality of positions, leaflets 12 or other soft body tissues of varying size and thickness can be stably grasped by the gripper arm 2030.

In use, the gripper control wire 2040 is configured to close the gripper arm 2030. When the gripper arm 2030 is closed, or grasping the leaflet 12 in the desired position, the gripper control wire 2040 is operated to move the link member 2052 into engagement with the lock member 2056. The first locking portion 2053, i.e. the teeth of the link member 20521, engages the second locking portion 2057, i.e. the ratchet gear rack. The ratchet gear rack prevents motion of the pawl (i.e. the link member 2052), such that the gripper arm 2030 is prevented from opening when the gripper lock mechanism 2050 is engaged.

FIGS. 14A-C show a gripper lock mechanism 2050 and its components in closer detail.

FIG. 14B shows the lock member 2056 in closer detail. In the present embodiment, the lock member 2056 comprises a large rack and a small rack 2056B. The small rack 2056B is mounted to the large rack 2056A via two pins 2056D, 2056E of 10 thou (0.254 mm) in diameter. The small rack 2056B comprises the second locking portion 2057, i.e. the plurality of teeth. The large rack 2056A facilitates the attachment of the small rack 2056B to the gripper housing 2006. The large rack 2056A can be welded or glued to the gripper housing 2006, and also comprises an opening 2056C to accommodate a hinged fixing of the proximal part 2004 of the housing to the distal part 2008 of the housing of the catheter device 2000. In other embodiments, the lock member 2056 can be formed of one single rack comprising the second locking portion 2057, for example as illustrated in FIG. 14.

FIG. 14C shows the link member 2052 in closer detail. The link member 2052 is connected to the gripper control wire 2040 via a pin 2054C of 18 thou (0.457 mm) in diameter at the second end 2054B. At the first end 2054A, and with reference to FIG. 15A, the link member 2054 is connected to the gripper arm 2030 via a pin or protrusion 2039 extending from the lever portion 2038.

FIGS. 15A and 15B illustrate two alternative arrangements for a catheter device 2000, according to embodiments of the present invention. In each arrangement there is provided an abutment portion 2055, 2059. The abutment portion 2055, 2059 is configured to prevent an overextension of the gripper arm 2030 that could otherwise cause the gripper arm 2030 to lock out in the open position, thus making motion of the gripper arm 2030 smoother. In each arrangement, the abutment portion 2055, 2059 prevents the first end 2054A (i.e. the end at which the link member 2052 is rotatably attached to the gripper arm 2030) crossing an axis or plane C-C′ extending between the second end 2054B of the link member 2052 and the hinge 2036 of the gripper arm 2030, during motion of the gripper arm 2030 between its opened and closed configurations.

Turning to FIG. 15A, the link member 2042 comprises an abutment portion 2055.

The abutment portion 2055 is a protrusion extending from the first end 2054A of the link member 2052. The abutment portion 2055 is configured to abut a surface, i.e. a shoulder region 2038A, of the lever portion 2038 when the gripper arm 2030 is opened. The abutment portion 2055 prevents the gripper arm 2030 from moving any further open when it abuts the lever portion 2038 of the gripper arm 2030. Accordingly, the abutment portion 2055 is configured to abut the shoulder region 2038A of the lever portion 2038 when the gripper arm 2030 is at its most open position, or in its most open configuration.

Turning to FIG. 15B, the abutment portion 2059 is a post or bar located in the gripper housing 2006. The abutment portion 2059 is configured to prevent the gripper arm 2030 from moving any further open by limiting the translation of the link member 2042, i.e. by abutting the link member 2052 when the gripper arm 2030 is opened.

FIG. 16 shows an alternative gripper lock mechanism 2050. The gripper lock mechanism 2050 differs by virtue of the link member 2052 being fixed to the gripper control wire 2040 via a hinge joint. The joint member 2042 of the gripper control wire 2040 comprises a cylinder member 2043 that engages the second end 2054B of the link member 2052 via a snap-fit engagement.

The link member 2052 also comprises an abutment portion 2055, which in the present embodiment as illustrated in FIG. 16 is a protrusion extending from the second end 2054B.

The abutment portion 2055 is configured to abut the joint member 2042 and thereby prevent an overextension of the gripper arm 2030.

FIGS. 17A to 17E show modified gripper lock mechanisms 2050. In each arrangement, the link member 2052 is rotatably attached to the gripper arm 2030 at the first end 2054A, but is instead fixed to the gripper control wire 2040 at the second end 2054B. As such, the link member 2052 does not rotate relative to the gripper control wire 2040.

The link member 2052 is attached to the gripper arm 2030 via the pin 2039 of the gripper arm extending from the lever portion 2038. The pin 2039 passes through an opening formed in the first end 2054A of the link member 2052. In the present embodiment, the opening is wider than the pin 2039, such that there is a gap or a clearance between the pin 2039 and the opening. Accordingly, the link member 2052 is configured to transfer a control input from the gripper control wire 2040 to the gripper arm 2030 with a degree of play or backlash. Such a motion may provide smoother control of the first locking portion 2053, when engaging the gripper lock mechanism 2053.

In some embodiments, the link member 2052 can be an integral component of the gripper control wire 2040. For example, the link member 2052 can be permanently fixed to the gripper control wire 2040 via welding or gluing. In the embodiment illustrated in FIG. 17A, the gripper control wire 2040 and the link member 2052 are formed as a single structure, such that the link member 2052 is a fixed part of the gripper control wire 2040. In other embodiments, the link member 2052 can be fixedly coupled to the gripper control wire 2040 via a fixed connection. For example, the link member 2052 can comprise a socket for receiving the gripper control wire 2040. In the embodiments illustrated in FIGS. 17B and 17C, the gripper control wire 2040 is fixedly connected to the link member 2052 via an interference fit.

In some embodiments the link member 2052 can be provided with a first locking portion 2053 having a single tooth or protrusion as shown in FIG. 17B, or in other embodiments the link member 2052 can be provided with a first locking portion 2053 having a plurality of teeth or protrusions, as shown in FIG. 17C. Providing multiple teeth can improve the resolution of the control of the link member 2052, since there is a plurality of stable positions for the link member 2052 to engage the lock member 2056.

FIGS. 17D and 17E show further modified arrangements for the gripper lock mechanism 2050. In each arrangement, the lock member 2056 comprises a first rack 2056D and a second rack 2056E. The first rack 2056D is used to fix the lock member 2056 to the proximal part 2004 of the housing of the catheter device 2000. The second rack 2056E comprises the second locking portion 2057 which, in the illustrated embodiments, comprise a plurality of teeth. The second rack 2056E is fixed to the first rack 2056D via a compliant mechanism 2056F, i.e. the second rack 2056E is compliantly fixed to the first rack 2056D. The second rack 2056E is resiliently biased to extend in the proximal direction.

In use, the gripper control wire 2040 can apply a force in the distal direction when closing the gripper arm 2030. In doing so, the link member 2052 may be forced into engagement with the second rack 2056E of the lock member 2056. The link member 2052 may cause the compliant mechanism 2056F to deform, thereby compressing the second rack 2056E in the distal direction. This motion alters the angle of the second locking portion 2057, such that the link member 2052 is able to slide along the lock member 2056. When the gripper arm 2030 is closed to the desired extent, the force applied by the gripper control wire 2040 can be relaxed. The compliant mechanism 2056F then causes the second rack 2056E to spring back, such that the first locking portion 2053 of the link member 2052 can engage the second locking portion 2057 of the lock member 2056.

Turning to FIG. 17D, in some embodiments the compliant mechanism 2056F comprises a flat spring formed by cutting the lock member 2056. In the present embodiment, the lock member 2056 is cut from a single piece of metal. The first rack 2056D and the second rack 2056E are hence integrally formed, or monolithic in structure.

FIG. 17E illustrates an alternative compliant mechanism 2056F. The compliant mechanism 2056F comprises a spring member extending between the first rack 2056D and the second rack 2056E. The second rack 2056E is connected to the first rack 2056D by the spring member and via a pivotal joint 2056G.

FIG. 18 shows an alternative gripper lock mechanism 2050 comprising a sprag clutch. The first locking portion 2053 of the link member 2052 comprises a sprag, and the second locking portion 2057 of the lock member 2056 defines a race. The sprag is shaped so as to backstop the link member 2052 when the sprag engages the race. The backstopping force of the sprag clutch is dependent on the frictional force experienced between the first locking portion 2053 and the second locking portion 2057. The gripper control wire 2040 generates a contact force Fn between the first locking portion 2053 and the second locking portion 2057. A frictional resistance Fr force is generated in a direction opposite to the direction of opening of the gripper arm 2030. In doing so, the gripper arm 2030 is prevented from opening, or at least the force required to inadvertently open the gripper arm 2030 during implantation of the anchor system is increased. Due to the shape of the sprag, the gripper lock mechanism 2050 resists the opening of the gripper arm 2030 rather than the closing of the gripper arm 2030.

The frictional force generated when the gripper lock mechanism is engaged depends on the frictional coefficients of the materials used to form the engaging surfaces of the first locking portion 2053 and the second locking portion 2057. Where steel is used for both locking portions 2053, 2057, the coefficient of friction is around 0.1 to 0.3. The frictional force can be increased by using suitable alternative materials.

In the present embodiment, the first locking portion 2053 of the link member 2052 comprises a single sprag. In other embodiments however, the first locking portion 2053 can comprise a plurality of sprags configured to each simultaneously engage the second locking portion 2057.

FIG. 19A shows another alternative gripper lock mechanism 2050 comprising a ratchet gear rack mechanism. The first locking portion 2053 is a toothed protrusion mounted to the link member via a spring member 2059A. The second locking portion 2057 is a ratchet gear rack.

The spring member 2059A is a curvilinear leaf spring. A first end of the spring member 2059A is connected to the link member 2052 towards the second end 2054B, and the first locking portion 2053 is located at an opposite second end of the spring member 2059A. In use, the gripper control wire 2040 applies a force in the distal direction that motivates the first locking portion 2053 into engagement with the second locking portion 2057. The spring member 2059A may introduce a degree of compliance that enables the first locking portion 2053 to more readily engage with the second locking portion 2057.

FIG. 19B shows a modified form of the link member 2052 of FIG. 20A. In this modified form, the first end of the spring member 2059A is connected to the second end 2054B of the link member 2052, and the second end of the spring member 2059A is connected to the first end 2054A of the link member 2052 via a hinge joint. The hinge joint comprises a cylindrical member 2059B mated via a snap-fit engagement to a socket 2059C. The hinge joint can provide additional support to the spring member 2059A during engagement of the first locking portion 2053 to the second locking portion 2057, since force will be transferred through both the spring member 2059A itself and the hinge joint when the gripper lock mechanism 2050 is engaged.

1. A catheter device for implanting an anchor system in soft body tissue, the catheter device comprising:
a gripper device for grasping soft body tissue, wherein the gripper device is configured to move between a closed configuration in which the soft body tissue is grasped and an opened configuration in which the soft body tissue is not grasped;
a gripper control member configured to move the gripper device between the closed configuration and the opened configuration; and
an anchor system deployment mechanism configured to deploy the anchor system into the soft body tissue when the soft body tissue is grasped by the gripper device;
wherein the gripper device comprises a gripper lock mechanism configured to releasably lock the gripper device in the closed configuration during deployment of the anchor system.

2. A catheter device as claimed in claim 1, wherein the gripper control member is configured to operate the gripper lock mechanism.

3. A catheter device as claimed in claim 1 or 2, wherein the gripper lock mechanism is configured to releasably lock the gripper device in one of a plurality of positions when in the closed configuration.

4. A catheter device as claimed in claim 1, 2 or 3, comprising:
a housing section, wherein the housing section extends from a proximal end to a distal end;
wherein the gripper device comprises a gripper arm coupled to the housing section.

5. A catheter device as claimed in claim 4, wherein the gripper lock mechanism comprises a link member connecting the gripper control member to the gripper arm, and a lock member fixedly attached to the housing section;
wherein the link member is configured to engage the lock member to releasably lock the gripper arm.

6. A catheter device as claimed in claim 5, wherein the lock member comprises a ratchet gear rack; and
wherein the link member comprises a protrusion configured to engage with the ratchet gear rack.

7. A catheter device as claimed in claim 6, wherein the link member comprises a plurality of protrusions configured to engage with the ratchet gear rack.

8. A catheter device as claimed in claim 5, 6 or 7, wherein a first end of the link member is rotatably coupled to the gripper arm, and a second end of the link member is rotatably coupled to the gripper control member.

9. A catheter device as claimed in claim 8, wherein the gripper lock mechanism comprises an abutment portion;
wherein the abutment portion is configured to prevent the first end of the link member crossing an axis extending between the second end of the link member and the centre of rotation of the gripper arm when the gripper arm is moved between the closed configuration and the opened configuration.

10. A catheter device as claimed in claim 9, wherein the abutment portion is a protrusion extending from the first end of the link member, wherein the protrusion is configured to abut a shoulder region of the gripper arm when the gripper arm is in the opened configuration.

11. A catheter device as claimed in claim 9, wherein the abutment portion is a post located in the housing section, wherein the post is configured to abut the link member when the gripper arm is in the opened configuration.

12. A catheter device as claimed in claim 5, 6 or 7 wherein a first end of the link member is rotatably coupled to the gripper arm, and a second end of the link member is fixed to the gripper control member.

13. A catheter device as claimed in claim 12, wherein the first end of the link member comprises an opening rotatably coupled to a pin of the gripper arm, wherein there is a clearance between the pin and the opening.

14. A catheter device as claimed in any preceding claim, wherein the gripper device comprises a plurality of serrations for grasping the soft body tissue.

15. A catheter device as claimed in any preceding claim, wherein the gripper device comprises an internal space configured to receive the anchor system during deployment of the anchor system.

16. A catheter device as claimed in any preceding claim, further comprising a soft tissue anchor system located within the anchor system deployment mechanism.

17. A catheter device as claimed in claim 16, wherein the soft tissue anchor system comprises a U-shaped fabric body comprising a base portion and at least two arm portions extending from the base portion, each arm portion being configured to collapse in folds towards the base portion such that, in use, the soft body tissue is sandwiched between the base portion and each of the arm portions.

18. A catheter device as claimed in claim 17, wherein each arm portion comprises an end cap fixed at an end of each arm portion distal to the base portion, and each end cap comprises an opening configured to engage a wire guide member for implanting the U-shaped fabric body in the soft body tissue, the catheter device optionally further comprising such a wire guide member for deploying the soft tissue anchor system.

19. A catheter device as claimed in any preceding claim, wherein the anchor system is a leaflet anchor system, the soft body tissue is a heart valve leaflet, and the catheter device is for implanting the leaflet anchor system in the heart valve leaflet to secure an artificial chordae line.

20. A catheter device as claimed in any of claims 1 to 17, comprising:
a two-part housing section extending from a proximal end of the catheter device along the length of the catheter device toward the distal end of the catheter device;
wherein the two-part housing section comprises a proximal part at the proximal end of the catheter device and a distal part located on the distal side of the proximal part;
wherein the gripper device, the gripper control member and a first anchor system deployment mechanism are located in the proximal part of the two-part housing section, wherein the first anchor system deployment mechanism is the anchor system deployment mechanism; and
wherein the distal part of the two-part housing section comprises a second anchor system deployment mechanism configured to deploy a second anchor system into further soft body tissue by moving the second anchor system outward in the distal direction relative to the distal part.

21. A catheter device as claimed in claim 20, wherein the first anchor system is a leaflet anchor system, wherein the soft body tissue is a heart valve leaflet, wherein the second anchor system is a papillary anchor system, and wherein the further soft body tissue is papillary muscle, and wherein the catheter device is for repair of the heart by implanting the leaflet anchor system and the papillary anchor system to secure an artificial chordae line.

22. A method of use of the catheter device of any preceding claim for repair of soft body tissue, the method comprising:
moving the gripper device from the closed configuration to the opened configuration;
bringing a surface of the gripper device into contact with the soft body tissue;
moving the gripper device from the opened configuration to the closed configuration so as to grasp the soft body tissue;
engaging the gripper lock mechanism; and
deploying the anchor system into the soft body tissue.
